(19) United States
(12) Patent Application Publication
(10) Pub. No.: US 2024/0402149 A1
(43) Pub. Date: Dec. 5, 2024

(54) AUTOMATED, MULTI-PARAMETER BLOOD-MEASURING SYSTEM

(71) Applicant: HEMETRIX, INC., San Diego, CA (US)

(72) Inventors: Matthew BANET, San Diego, CA (US); Ali NSAIR, Westchester, CA (US)

(73) Assignee: HEMETRIX, INC., San Diego, CA (US)

(21) Appl. No.: 18/658,902

(22) Filed: May 8, 2024

Related U.S. Application Data
(60) Provisional application No. 63/500,894, filed on May 8, 2023.

(51) Int. Cl. G01N 33/49; G01N 21/31; G01N 21/55; G01N 27/06; G06T 7/00; G06T 7/20

(52) U.S. Cl. CPC G01N 33/4905; G01N 21/31; G01N 21/55; G01N 27/06; G06T 7/0012; G06T 7/20; G06T 2207/20081; G06T 2207/30004

(57) ABSTRACT

The invention provides a system for automatically measuring an Activated Clotting Time value and other parameters from a sample of blood.

### CROSS-REFERENCE TO RELATED APPLICATIONS

The present invention claims the benefit of priority to U.S. Provisional Application Ser. No. 63/500,894, filed on May 8, 2023, which is incorporated herein by reference in its entirety.

### BACKGROUND AND FIELD OF THE INVENTION

### Field of the Invention

The invention relates to the fields of aspirating blood from patients and then measuring properties of the blood during surgical procedures and post-surgery recovery.

### General Background

Cardiovascular disease is the one of largest causes of morbidity and mortality worldwide. During the last few decades, this trend has driven an evolution and rapid growth of: 1) percutaneous coronary interventions (herein “PCI”) procedures that feature coronary stenting for clearing arteries that are blocked due to atherosclerotic disease; and 2) minimally invasive procedures that feature percutaneous trans-catheter aortic valve replacements (herein “TAVR”) for aortic valve disease and leaking mitral valves.

Both PCI and TAVR procedures are typically done in a hospital's catheterization lab (herein “Cath Lab”) under the cover of heparin, a ubiquitous anticoagulant that prevents formation of blood clots in both the patient and catheters used for these procedures. Heparin is carefully dosed, usually based on the patient's weight, to maintain the patient within a narrow therapeutic anticoagulation range designed to avoid both blood clot formation and severe, potentially fatal bleeding. However, heparin is a heterogeneous molecule that has varying degrees of activity and affects each patient differently. This means coagulation within the patient must be monitored frequently to balance the risks of clot formation and excessive blood thinning that can cause catastrophic bleeding. In the Cath Lab, coagulation is typically characterized by measuring the patient's activated clotting time (herein “ACT”), which represents the time blood takes to clot. ACT is currently measured during procedures with a standard point of care (herein “POC”) device. Typically, ACT is between 150-350 seconds. It is typically measured throughout PCI and TAVR procedures (e.g. every 15 minutes or so, typically resulting in 1-15 measurements/procedure). It is almost always measured immediately before and after the procedure's stent and balloon catheter are deployed.

During a typical PCI or TAVR procedure, a nurse initiates an intravenous (herein “IV”) catheter into a vein in the patient's arm. Medications (e.g. sedatives) and heparin are delivered to the patient through the IV. Clinicians then clean an area and administer a local anesthetic where the procedure takes place, typically in an artery in the patient's wrist or groin. An incision is then made into the skin over the artery, and the cardiologist inserts a sheath through the incision site and into the artery. Once the sheath is in the vessel, the cardiologist guides a catheter into it through the sheath. Using a live X-ray, the cardiologist threads the catheter through the artery into the heart. There, the cardiologist injects a contrast dye to determine how well the heart is functioning. Through this process, called angiography, the cardiologist visually identifies blood vessels in the heart, along with any blockages or narrowing. The procedure typically lasts 30-60 minutes.

The PCI procedure typically follows angiography, during which the cardiologist inserts into the artery a catheter featuring a balloon mounted to its tip and a stent attached to the balloon. Using images taken during angiography, the cardiologist positions the stent in the coronary artery featuring blockage. At the location of the blockage, the cardiologist inflates the balloon, which widens the artery, stretching it so blood flow can return to normal. Sometimes a cardiologist inserts the stent at the site to keep the artery from narrowing in the future. After the artery is widened, the cardiologist deflates the balloon and removes the catheter. ACT is measured throughout this procedure.

Coagulation is also monitored from patients recovering from surgeries and receiving heparin drips. Here, clinicians measure a parameter called partial thromboplastin time (herein “PTT”) or activated partial thromboplastin time (herein “aPTT”) between 1-5 times/day during the patient's entire hospital stay, which averages 5 days. PTT has a normal range between 10-15 seconds, and is typically considered to be a better indicator of a patient's clotting factors compared to ACT; it measured in the hospital's hematology lab. PTT is not used in the Cath Lab, mostly because of convenience and the time required for the assay that measures it.

Current POC devices for measuring ACT and other parameters such as hemoglobin are remote from the patient and require a clinician to: 1) stop the procedure and aspirate a blood sample (typically 3-5 ccs) with a syringe to confirm the patient is adequately anticoagulated before commencing the procedure; 2) disconnect the syringe from the patient and manually transport it to the POC device; and 3) measure the sample with the device over a time period of 5-7 minutes. Each of these steps are associated with multiple potential complications and adverse events, such as delays, introduction of air bubbles during aspiration, and formation of blood clots that may travel to the heart (and possibly induce cardiac arrest) or brain (stroke). Often the surgeon, focused on the highly technical procedure at hand, forgets to initiate an ACT measurement. Furthermore, if a patient is under-dosed on heparin, the physician may need to increase this dose, wait five minutes for a follow-on test, and then repeat the measurement. And finally, the blood sample used to test for ACT and H&H is withdrawn from the same catheter used to deliver the stent and other equipment. This has the potential to introduce in-procedure complications, such as air entrapment. In short, the current standard of care for ACT measurements is fraught with many problematic issues, a situation that means it is common for cardiac surgeons to forego testing in critical clinical situations.

Additionally, most POC devices for measuring ACT do not simultaneously measure other blood-based parameters, such as hemoglobin; this typically requires a completely separate POC device. Simultaneous measurement of hemoglobin and ACT offers a very significant advantage for monitoring blood levels and any other signs of bleeding in long, complex procedures, such as open-heart surgeries. Not having to withdraw 3-5 cc blood every 5 to 15 minutes will save the patient unnecessary blood loss; this is particularly beneficial for anemic patients and, perhaps even more importantly, during future use in pediatric patients.

POC devices for measuring ACT include Abbott's I-STAT, Medtronic's ACT Plus, and Hemachron's Signature Elite, each of which is purchased by hospitals and reimbursed by private and non-private payers. Currently, none of these systems make continuous, automated measurements of ACT and hemoglobin. Each requires discrete blood samples that a clinician measures manually and multiple times during a procedure; a disposable test cartridge (typically costing $5-10) is used and disposed of after each test.

Each year, there are about 1 million PCI procedures and 113,000 TAVRs performed in the United States. The PCI procedures, with an average published cost of $118,000/procedure, represent an estimated market of $15 billion. 13% of these procedures feature bleeding-related complications, which prolongs the procedure, incurs additional costs (adding roughly $30,000/procedure), and increases the risk of morbidity and mortality. Every PCI and TAVR procedure is conducted under the cover of heparin with patients featuring similarly narrow therapeutic ACT ranges.

Beyond minimally invasive cardiac interventions, ACTs are also measured in operating rooms for open heart surgeries during cardiopulmonary bypass (32.4% of reporting sites), cardiac catheterization (32.3%), intensive or coronary care unit (13.8%), vascular surgery or catheterization (10.1%), hemodialysis (1.1%), and other procedures (10.2%). Methodologies for measuring ACTs—especially those that cure the well-defined deficiencies of these measurements, as described below—can thus have wide-ranging case use.

Heparin and subsequent ACT measurements are also used during neurology procedures, which include thrombolytic therapy, embolization of intracranial and head/neck aneurysms, cerebral angiography, and carotid artery angioplasty/stenting. In 2021, approximately 310,000 of these procedures were performed domestically.

In 2021, there were approximately 2M post-surgical patients receiving heparin drips and undergoing PTT measurements every few hours during their hospital stay.

The market for coagulation analyzers market alone (which includes ACT-measuring devices) was $4.84 Billion in 2021, up from $2.98 Billion in 2016, at a CAGR of 10.2% from 2016 to 2021. Factors driving growth of this market are an aging population, increasing prevalence of autoimmune diseases, and increasing volume of procedures requiring hematologic monitoring.

### SUMMARY OF THE INVENTION

Based on the above, it would be beneficial to have a ‘Smart Syringe Measurement Device’ (herein “SSMD”) that automatically aspirates blood from a patient and measures important parameters therefrom, e.g. ACT, PTT, aPTT, PT, thrombin, others (herein “coagulation parameters); creatinine, glucose, cortisol, lactate, lactic acid, brain natriuretic peptide (herein “BNP”), others (herein “biomarkers”); hemoglobin, hematocrit, pH others (herein “blood parameters”); and sodium, potassium, calcium (herein “blood-based ions”). Candidates for the SSMD are patients undergoing cardiac and neurological procedures, and those recovering post-surgery.

The SSMD's measurement system features a collection of sensors disposed within a compact electronics module. This includes an innovative marriage of: 1) optical, impedance, and mechanical sensors; 2) machine-learning algorithms; and 3) automation. Collectively, these sensors measure coagulation parameters, biomarkers, blood parameters, and blood-based ions from the patient. With the SSMD, the surgeon can receive real-time, critical information and focus on the highly technical procedure at hand, with fewer interruptions, improved procedural turn-around time that minimizes human error. In embodiments, the surgeon programs a ‘profile’ of measurements into the SSMD with a touchpanel display; the system then automatically makes these measurements and displays results without any further human interaction. This approach features additional benefits of reducing volumes of manual blood aspirations and thus the risk of potentially catastrophic air or blood clots entering the patient. Additionally, the SSMD can be integrated into a closed-loop system that provides real-time measurements of critical biomarkers and rapid response to derangements to maximize efficiency and remove human error and delays. In embodiments, for example, the SSMD makes automated, real-time measurements of coagulation parameters, and then delivers heparin to the patient through a closed-loop system.

In summary, the SSMD offers the following value propositions: (1) reducing complication risks, time and resource utilization, and costs of PCIs, TAVRs, and other interventional procedures through continuous, on-patient monitoring of coagulation parameters, blood parameters, and in certain instance biomarkers and ions; (2) reducing complications of interventions related to arterial air bubbles and embolic clotting, a potentially lethal side effect that can cause a heart attack, stroke, or death; (3) improving resource utilization and automated completion of tests for coagulation and blood parameters in critical clinical situations; and (4) ultimately demonstrating a positive effect on patient outcomes.

In one aspect, the invention provides a system for predicting ACT from a blood sample within a sample holder. The system features a first ACT-measuring system that measures a mechanical property indicating clotting of the blood sample, and a second ACT-measuring system that measures first and second time-dependent waveforms that are both affected by clotting of the blood sample. A processing system receives information from these systems and then performs the following steps (e.g. using computer code): 1) analyze the mechanical property to determine a first value of ACT; 2) analyze both the first time-dependent waveform and the first value of ACT to determine a model for predicting ACT; and 3) using the model, analyze the second time-dependent waveform to predict a second value of ACT.

In embodiments, the first ACT-measuring system includes a digital camera that images the blood sample, and a mechanical system that moves the sample holder. For example, the mechanical system can include a vibrator system coupled to the sample holder that rapidly moves it, e.g. vibrates it. Here, step 1) performed by the processing system features analyzing motion of blood clots within the sample holder to determine the first value of ACT. Step 1) performed by the processing system can use an algorithm based on, for example, pattern recognition, machine learning, artificial intelligence, or related computation techniques to analyze motion of blood clots within the sample holder.

In related embodiments, the sample holder includes reflective beads or similar materials that are mixed with the blood sample. Here, step 1) performed by the processing system involves analyzing motion of the reflective beads to determine the first value of ACT using the computational techniques described above.

In other related embodiments, the mechanical system includes a motorized system that is connected to the sample holder and configured to move it. The motorized system, for example, can rock the sample holder back and forth, or move it in a similar manner. Here, the digital camera can collect images of blood moving within the sample holder, and then step 1) performed by the processing system can analyze motion of the blood within the sample holder to determine the first value of ACT. For example, the digital camera can collect images of a blood/air interface within the sample holder, and then step 1) analyzes the blood/air interface to determine the first value of ACT. As before, the processing system can perform this analysis using computational techniques such as pattern recognition, machine learning, and artificial intelligence to analyze the blood/air interface.

In embodiments, the second ACT-measuring system features an optical system, e.g. one that includes a light source and a photodetector. The light source can be positioned on one side of the sample holder, and the photodetector can be positioned on an opposing side of the sample holder. In this manner, the light source and photodetector measure time-dependent optical absorption of the blood sample to determine the first time-dependent waveform. Then step 2) performed by the processing system involves analyzing the time-dependent optical absorption of the blood sample and the first value of ACT to determine the model for predicting ACT. As with step 1) described above, step 2) can use an algorithm based on numerical fitting, pattern recognition, machine learning, and artificial intelligence to analyze the blood/air interface. Alternatively, the light source and the photodetector are positioned on the same side of the sample holder, and step 2) includes similar algorithmic processing techniques.

In other embodiments, the second ACT-measuring system includes an impedance/reactance system. Such a system, for example, can include a sense electrode and a drive electrode, wherein the drive electrode injects electrical current into the blood sample, and the sense electrode measures a voltage that is a function of the injected electrical current. Here, the impedance/reactance system measures time-dependent electrical impedance of the blood sample to determine the first time-dependent waveform. Then, in a manner similar to that used with the optical system, step 2) performed by the processing system involves analyzing the time-dependent electrical impedance of the blood sample and the first value of ACT to determine the model for predicting ACT. As before, step 2) in this cases uses algorithms based on numerical fitting, pattern recognition, machine learning, and artificial intelligence to analyze the blood/air interface. In place of time-dependent electrical impedance, this same system can measure time-dependent electrical reactance of the blood sample to determine the first time-dependent waveform, and then process it as described above.

In another aspect, the invention provides a system for predicting ACT from a blood sample within a sample holder that includes a first ACT-measuring system featuring an imaging system that measures a mechanical property indicating clotting of the blood sample; a second ACT-measuring system featuring an optical system that measures first and second time-dependent waveforms that are both affected by clotting of the blood sample; and a processing system that performs the following steps: 1) analyze the mechanical property to determine a first value of ACT; 2) analyze both the first time-dependent waveform and the first value of ACT to determine a model for predicting ACT; and 3) using the model, analyze the second time-dependent waveform to predict a second value of ACT.

In another aspect, then invention provides a system for measuring a parameter from a blood sample from a patient that includes the following components: 1) an automated blood-extraction component connected to the patient and featuring a motorized system that automatically extracts the blood sample from the patient; 2) a sample cuvette coupled to the automated blood-extraction component that receives the blood sample; 3) an automated sample-handling system that moves the sample cuvette; 4) a centrifuge system that receives the sample cuvette from the automated sample-handling system and centrifuges it; 5) a measurement component that receives the sample cuvette from the automated sample-handling system after it has been centrifuged by the centrifuge system and measures a signal from the blood sample; and 6) a processing system configured to process the signal to determine the parameter.

In embodiments, the automated blood-extraction component features a catheter inserted into a blood vessel (e.g. an artery or a vein) within the patient. The automated blood-extraction component can, for example, include a motorized pump that pumps the blood sample from the blood vessel into the sample cuvette.

In other embodiments, the measurement component features a digital camera, and the signal is an image of the blood sample. Here, the processing system processes the image with an algorithm, e.g. one based on pattern recognition, machine learning, and artificial intelligence, to analyze the image. For example, the algorithm can estimate a volume of plasma and a volume of blood cells from the image, and then use these parameters to estimate a value of hematocrit.

In other embodiments, the measurement system features an optical system, e.g. one that includes a light source and a photodetector. Here, the signal is a time-dependent waveform, with the light source generating a beam of radiation, and the photodetector detecting the beam of radiation after it passes through the blood sample to generate the time-dependent waveform. The processing system can then process the time-dependent waveform with an algorithm, based on similar computational techniques described above, to process the time-dependent waveform to estimate the clotting time.

In a related aspect, the invention includes all the same components as described above, along with an automated pipetting system (e.g. one including a digital camera) that can process the image and, based on the processing, extract a portion of plasma, serum, and/or blood cells from the first sample cuvette and deposit the portion into a second sample cuvette from which the parameter is measured.

In embodiments, the system further includes a reservoir that contains a reagent, and the automated pipetting system extracts a volume of reagent from the reservoir, and then deposits it in the second sample cuvette. Here, for example, the measurement component may include an optical system (e.g. one with a light source and a photodetector) that measures an optical absorption spectrum from a mixture of the volume of reagent and the portion of plasma, serum, and/or blood cells within the second sample cuvette. In related embodiments, the automated pipetting system selects plasma from the first sample cuvette, the volume of reagent comprises a clotting agent, and the measurement component processes the optical absorption spectrum measured by the optical system to estimate either aPTT and PTT. Or the automated pipetting system selects plasma from the first sample cuvette, the volume of reagent comprises hexokinase, glucose-6-phosphate dehydrogenase, and/or NAD+, and the measurement component processes the optical absorption spectrum measured by the optical system to estimate glucose. Or the automated pipetting system selects serum from the first sample cuvette, the volume of reagent comprises sodium borate and/or picric acid, and the measurement component processes the optical absorption spectrum measured by the optical system to estimate creatinine. Or the automated pipetting system selects serum from the first sample cuvette, the volume of reagent comprises an aptamer that binds to thrombin, and the measurement component processes the optical absorption spectrum measured by the optical system to estimate thrombin. Or the automated pipetting system selects plasma from the first sample cuvette, the volume of reagent comprises an aptamer that binds to lactic acid, and the measurement component processes the optical absorption spectrum measured by the optical system to estimate lactic acid. Or the automated pipetting system selects serum from the first sample cuvette, the volume of reagent comprises Bromcresol Purple, and the measurement component processes the optical absorption spectrum measured by the optical system to estimate albumin. And finally, the automated pipetting system selects blood cells from the first sample cuvette, the volume of reagent comprises Drabkin's reagent, and the measurement component processes the optical absorption spectrum measured by the optical system to estimate hemoglobin.

In another aspect, the invention provides a system for measuring a concentration of an ionic compound from a patient's blood sample. The system includes: 1) an automated blood-extraction component connected to the patient featuring a motorized system that automatically extracts the blood sample from the patient; 2) a first sample cuvette coupled to the automated blood-extraction component that receives the blood sample; 3) an automated sample-handling system that moves the first sample cuvette; 4) a centrifuge system that receives the first sample cuvette from the automated sample-handling system and centrifuges the blood sample to separate it into at least one of plasma and blood cells; 5) a measurement component featuring a digital camera that receives the first sample cuvette from the automated sample-handling system after it has been centrifuged by the centrifuge system and captures an image from the blood sample, the imaging showing separated plasma and blood cells; 6) an automated pipetting system that processes the image and, based on the processing, extracts a portion of the plasma from the first sample cuvette and deposit it into a second sample cuvette; and 7) an ion-specific electrode in contact with the plasma in the second sample cuvette that measures the ionic compound therefrom.

In embodiments, the automated blood-extraction component includes a catheter inserted into a blood vessel within the patient, and a motorized pump that aspirates the blood sample from the blood vessel.

In other embodiments, the system includes a voltage-measuring system in electrical contact with the ion-specific electrode. The measurement component typically includes a processing system that operates an algorithm that processes a signal from the voltage-measuring system to measure the concentration of the ionic compound. For example, the algorithm can include a look-up table that correlates a collection of voltage values to concentrations of the ionic compound.

The ion-specific electrode can be configured to specifically measure hydrogen ions, and then the algorithm converts a value corresponding concentration of hydrogen ions into a value of pH. Alternatively, the system includes one or more ion-specific electrodes configured to specifically measure potassium, chlorine, chloride, calcium, and/or sodium ions.

In another aspect, the system includes all the same components as described above, plus a sensor coupled to a third sample cuvette and configured to measure the additional parameter therefrom. The system, for example, may further additionally include a reservoir that contains a reagent. Here, the automated pipetting system can extract a volume of reagent from the reservoir, and then deposit the volume of reagent into the third sample cuvette. The measurement component may include an optical system, e.g. one with a light source and a photodetector, that measures an optical absorption spectrum from a mixture of the volume of reagent and the blood cells within the third sample cuvette. In embodiments, the volume of reagent comprises Drabkin's reagent, and the measurement component processes the optical absorption spectrum measured by the optical system to estimate hemoglobin.

In another aspect, the invention features a system for measuring ACT and an additional parameter from a blood sample from a patient featuring the following components: 1) a sample cuvette that receives the blood sample; 2) a motorized cuvette-moving system, attached to the sample cuvette, and that moves the sample cuvette from a first position to a second position; 3) an imaging system including a digital camera that collects at least one image from the blood sample within the sample cuvette as the motorized cuvette-moving system moves the sample cuvette from the first position to the second position; 4) an optical absorption system featuring a light source and a photodetector, with the light source positioned to emit optical radiation that passes through the blood sample as the motorized cuvette-moving system moves the sample cuvette from the first position to the second position, and the photodetector positioned to detect the optical radiation after it passes through the blood sample and generate a signal; and 5) a processing system operating computer code that: A) processes the image with a first algorithm to determine a value of ACT from the blood sample; and B) processes the signal with a second algorithm to determine a value of the additional parameter from the blood sample.

In embodiments, the motorized cuvette-moving system changes an angle of the sample cuvette relative to a vertical axis. For example, the first position the sample cuvette can be vertical, and in the second position the sample cuvette is angled relative to vertical. Here, the imaging system captures a first image of the blood sample while the sample cuvette is vertical, and a second image of the blood sample when the sample cuvette is angled relative to vertical. The first algorithm then calculates a difference in the first and second images to measure the ACT. In embodiments, both the first and second images are images of a blood/air interface, with differences in this interface indicating the ACT. Alternatively, the first and second images are images of a blood clot within the blood sample, and the first algorithm calculates movement of the blood clot to measure the ACT. As with similar systems described herein, the algorithm can be based on pattern recognition, machine learning, artificial intelligence, or any other computational technique described herein.

In embodiments, the signal indicates an optical absorption property of the blood sample, e.g. an optical absorption spectrum or a time-resolved waveform. For example, the optical absorption system can include a bandpass optical filter positioned in front of the photodetector, and the bandpass optical filter can be automatically controlled by a computer.

In related aspects, the system is similar to that described above, but instead of the optical system, the system includes an impedance/reactance system featuring a sense electrode and a drive electrode, with the drive electrode configured to pass an electrical current through the blood sample as the motorized cuvette-moving system moves the sample cuvette from the first position to the second position, and the sense electrode configured to detect a signal indicating at least one of a change in electrical resistance and reactance of the blood sample in response to the electrical current.

Here, in embodiments, the signal indicates an electrical property that is, e.g., impedance, capacitance, reactance, an electrical resonant frequency, and/or resistance property of the blood sample. The electrical property, for example, can be a time-resolved waveform, and the second algorithm calculates the additional parameter from the electrical property.

In another aspect, the invention provides a system for measuring ACT and an additional parameter from a blood sample from a patient. The system includes the following components: 1) a sample cuvette that receives the blood sample and an external component; 2) a motorized cuvette-vibrating system, attached to the sample cuvette, and that vibrates the sample cuvette containing the blood sample and external component therein; 3) an imaging system featuring a digital camera that collects one or more images from the blood sample and external component within the sample cuvette as the motorized cuvette-vibrating system vibrates the sample cuvette; 4) an optical absorption system featuring a light source and a photodetector, with the light source positioned to emit optical radiation that passes through the blood sample as the motorized cuvette-vibrating system vibrates the sample cuvette, and the photodetector positioned to detect the optical radiation after it passes through the blood sample and generate a signal; and 5) a processing system configured to: A) process at least one image with a first algorithm to determine a value of ACT from the blood sample; and B) process the signal with a second algorithm to determine a value of the additional parameter from the blood sample.

In embodiments, the motorized cuvette-vibrating system vibrates the sample cuvette at a frequency of between 20-250 Hz. The imaging system captures a first image of the blood sample at one point in time and a second image of the blood sample at a second point in time, with both the first and second images captured while the sample cuvette is vibrating. The first algorithm, which is typically based on the numerical techniques described herein, calculates a difference in the first and second images to measure the ACT.

In embodiments, the external component is a collection of reflective beads, and the first algorithm calculates a parameter indicating collective movement of the reflective beads. The parameter, for example, can be an ensemble average. For example, the first algorithm can be configured to process the ensemble average to determine a reduction in movement of the reflective beads, with a time associated with the reduction in movement indicating ACT. In other embodiments, the parameter is a first time-domain waveform, and the first algorithm is further configured to process the time-domain waveform to determine a reduction in movement of the reflective beads, with a time associated with the reduction in movement indicating ACT. For example, the first algorithm can calculate a parameter indicating movement of one of the reflective beads. Here, the first algorithm is further configured to process the time-domain waveform to determine a reduction in movement of the reflective beads, with a time associated with the reduction in movement indicating ACT.

In other embodiments, the first and second images show a blood clot within the blood sample, and the first algorithm calculates movement of the blood clot to measure the ACT. In embodiments, the signal indicates an optical absorption property of the blood sample, e.g. an optical absorption spectrum or a time-resolved waveform.

In other aspects, the invention provides a similar system for measuring ACT, but in place of the optical system is an impedance/reactance system that features a sense electrode and a drive electrode, with the drive electrode configured to pass an electrical current through the blood sample as the motorized cuvette-vibrating system vibrates the sample cuvette, and the sense electrode configured to detect a signal indicating at least one of a change in electrical resistance and reactance of the blood sample in response to the electrical current. Here, the signal indicates an electrical property that is one of an impedance, capacitance, reactance, an electrical resonant frequency, and/or resistance property of the blood sample. For example, the electrical property can be a time-resolved waveform, and the second algorithm calculates the additional parameter from the electrical property.

In a related aspect, the invention provides a system with similar components (sample cuvette, mechanical system, digital camera, optical system, and processing system) for measuring a property from a blood sample, e.g. a coagulation property. The coagulation property is either ACT, PTT, aPTT, and/or PT.

In embodiments, the mechanical system translates the sample cuvette so that its vertical axis is moved in a time-dependent manner. Images collected by the digital camera show clotting blood, and the processing system processes the images to determine motion of the clotting blood within the sample cuvette. Alternatively, the mechanical system rotates the sample cuvette along an axis, or alternatively vibrates the sample cuvette, and again the images collected by the digital camera show clotting blood that the processing system processes to determine motion of the clotting blood within the sample cuvette. Alternatively, the sample cuvette includes a loose mechanical component, such as a ball, rod, particle, disk, rotor, or a mechanical version thereof, that becomes surrounded by blood when the sample cuvette receives the blood sample. The mechanical system can then translate, rotate, or vibrate the sample cuvette, and the images collected by the digital camera show the loose mechanical component. The processing system then processes the images to determine motion of the blood sample and the loose mechanical component within the sample cuvette. Finally, these images are analyzed with an algorithm to determine the parameter.

In another aspect, the invention provides a system for delivering fluids to a patient (e.g. a closed-loop system). The system includes: 1) an automated blood-extraction component connected to the patient and featuring a motorized system that automatically extracts the blood sample from the patient; 2) a sample cuvette coupled to the automated blood-extraction component that receives the blood sample; 3) an automated sample-handling system that moves the sample cuvette; 4) a centrifuge system that receives the sample cuvette from the automated sample-handling system and centrifuges it; 5) a measurement component that receives the sample cuvette from the automated sample-handling system after it has been centrifuged by the centrifuge system and measures a value of hematocrit from the blood sample; and 6) a fluid-delivery system that processes the value of hematocrit and, in response, deliver a volume of fluids to the patient.

In embodiments, the automated blood-extraction component comprises a catheter inserted into a blood vessel within the patient and a motorized pump that pumps the blood sample from the blood vessel into the sample cuvette. The measurement component is typically a digital camera, and the signal is an image of the blood sample within the sample cuvette as measured by the digital camera. The processing system processes the image with an algorithm, e.g. one based on pattern recognition, machine learning, and artificial intelligence, that analyzes the image. With this approach, the measurement component estimates a volume of plasma and a volume of blood cells from the image, and by collectively processing these calculates the value of hematocrit. Then the fluid-delivery system processes the value of hematocrit with an algorithm to determine a volume of fluids to deliver to the patient. For example, the algorithm can be a look-up table that correlates values of hematocrit to volumes of fluids; alternatively, it can be something more sophisticated, e.g. an algorithm based on machine learning. In both cases, it can additionally process biometric information from the patient to determine a volume of fluids to deliver to the patient.

In embodiments, the blood-extraction system and fluid-delivery system are coupled together. For example, they can both use a common catheter.

In another aspect, the invention provides a similar system, only it measures hemoglobin from the blood sample, and then the fluid-delivery system process the value of hemoglobin to deliver a volume of fluids to the patient. Typically, hemoglobin is measured with an optical system like that described herein, wherein the optical system measures an absorption spectrum from the blood sample. In embodiments, the blood sample is a mixture of red blood cells and a reagent, wherein the reagent reacts with the hemoglobin, thereby yielding an absorption spectrum with features (e.g. peaks) that vary with the amount of hemoglobin. Similar to before, the fluid-delivery system processes the value of hemoglobin (alone, or collectively with biometric information corresponding to the patient) with a second algorithm to determine a volume of fluids to deliver to the patient.

In another aspect, the invention provides a method for monitoring a coagulation parameter from a patient, comprising the following steps, all performed automatically with a computer-controlled system: 1) aspirating a blood sample from the patient with a pump connected to a catheter inserted in a blood vessel within the patient; 2) porting the blood sample to a sample cuvette; 3) mixing the blood sample with a clotting agent within the sample cuvette; 4) measuring a clotting time from a mixture containing the blood sample and the clotting agent; and 5) displaying the clotting time.

In this method, the pump can be a computer-controlled syringe pump, and step 1 further includes using a first motor and software program running on a computer to activate the syringe pump to automatically aspirate the blood sample. For example, the computer-controlled syringe pump can include a syringe connected to the first motor, and the software program running on a computer activates the first motor to draw back a plunger within the syringe. In embodiments, the catheter includes a lumen (preferably positioned within the catheter) that automatically aspirates the blood sample. The system can also include a computer-controlled pump that connects to the lumen. Then, step 1 can include aspirating blood through the lumen with the computer-controlled pump. Alternatively, the lumen connects to a second motor, and step 1 further includes using a software program running on a computer to activate the second motor to push the lumen into the blood vessel.

In embodiments, step 1 can additionally include the following steps, all performed automatically with the computer-controlled system: 1A) pushing the lumen into the patient's blood vessel; and 1B) aspirating the blood sample through the lumen. Step 1A can also include pushing the lumen a pre-determined distance into the patient's blood vessel, where the pre-determined distance is stored in a memory in the computer-controlled system. Step 1 can then include aspirating the blood sample from the patient with the pump connected to the catheter inserted in a vein within the patient.

In embodiments, step 3 further includes mixing the blood sample with the clotting agent within the sample cuvette using a centrifuge. For example, step 3 can include the following steps, all performed automatically with the computer-controlled system: 3A) loading the sample cuvette with the blood sample in centrifuge; and 3B) activating the centrifuge to mix the blood sample with the clotting agent.

In other embodiments, the method additionally includes the following steps, all performed automatically with the computer-controlled system: 6) after steps 1-5, flushing the catheter with a solution; and 7) repeating steps 1-5. For example, the method can include repeating steps 6 and 7 according to a schedule programmed into the computer-controlled system. The method can also include step 8, which involves displaying the parameter (e.g. ACT) on a display that can be easily viewed by the surgeon.

In another aspect, the invention provides a method for monitoring an amount of hemoglobin from a patient, comprising the following steps, all performed automatically with a computer-controlled system: 1) aspirating a blood sample from the patient with a pump connected to a catheter inserted in a blood vessel within the patient; 2) porting the blood sample to a sample cuvette; 3) optically measuring a signal from the blood sample within the sample cuvette; 4) processing the signal to estimate the amount of hemoglobin; and 5) displaying the amount of hemoglobin.

In embodiments, the pump is a computer-controlled syringe pump, and step 1 further includes using a first motor and software program running on a computer to activate the syringe pump to automatically aspirate the blood sample. The computer-controlled syringe pump typically includes a syringe connected to the first motor, and the software program running on a computer activates the first motor to draw back a plunger comprised by the syringe. In embodiments, the catheter includes a lumen (e.g. in its interior) that automatically aspirate the blood sample. For example, in embodiments a computer-controlled pump connects to the lumen, and step 1 includes aspirating blood with the computer-controlled pump through the lumen. Alternatively, the lumen connects to a second motor, and step 1 further includes using a software program running on a computer to activate the second motor to push the lumen into the blood vessel. For example, step 1 can include the following steps, all performed automatically with the computer-controlled system: 1A) pushing the lumen into the patient's blood vessel; and 1B) aspirating the blood sample through the lumen. In embodiments, step 1A further includes pushing the lumen a pre-determined distance into the patient's blood vessel, where the pre-determined distance is stored in a memory in the computer-controlled system. Then step 1 includes aspirating the blood sample from the patient with the pump connected to the catheter inserted in a vein within the patient.

In embodiments, after step 2 and before step 3, the method further includes mixing the blood sample with a reagent, such as Drabkin's reagent, or a chemical derivative thereof.

In other embodiments, the method further includes the following steps, all performed automatically with the computer-controlled system: 6) after steps 1-5, flushing the catheter with a solution; and 7) repeating steps 1-5. As before, the method can include repeating steps 6 and 7 according to a schedule programmed into the computer-controlled system. The schedule, for example, can be selected from a menu that is viewable on a graphical user interface previously programmed into the computer-controlled system.

In another aspect, the invention provides a similar method for monitoring a value of hematocrit from a patient. Step 1) is similar to that described above. The additional steps of this method are: 2) porting the blood sample to a sample cuvette; 3) porting the sample cuvette to a centrifuge; 4) activating the centrifuge to spin the sample cuvette to separate red and white blood cells from plasma within the blood sample; 5) measuring an image of the red and white blood cells and plasma within the sample cuvette with a camera; 6) processing the image with an algorithm to estimate the value of hematocrit; and 7) displaying the value of hematocrit.

In related aspects, the invention provides a method for measuring both hemoglobin and hematocrit using a combination of the steps described above.

And in yet another aspect, the invention provides a system for measuring a set of parameters from blood samples from a patient. The system includes the following components: 1) a computer system that receives input from a user describing a sequence of multiple measurements; 2) an automated blood-extraction component connected to the patient and controlled by the computer system and that includes a motorized system to automatically extract a first blood sample from the patient; 3) a first sample cuvette coupled to the automated blood-extraction component that receives the first blood sample; 4) an automated sample-handling system controlled by the computer system and that moves the first sample cuvette from one location to another; 5) a measurement component controlled by the computer system that receives the first sample cuvette from the automated sample-handling system and measures a first signal from the first blood sample; and 6) a processing system controlled by the computer system that processes the first signal to determine a first parameter in the set of parameters.

In embodiments, the automated blood-extraction component automatically extracts a second blood sample from the patient after extracting the first blood sample from the patient. The automated blood-extraction component includes a catheter connected to a motor-controlled pump, wherein the computer system is in electrical contact with the motor-controlled pump. In embodiments, the computer system receives inputs from the user that dictate volumes of the first and second blood samples. The system can include a second sample cuvette coupled to the automated blood-extraction component that receives a second blood sample.

An area within the system (e.g. a cassette) stores the first and second cuvettes. For example, the area can include a set of sample cuvettes, with each cuvette in the set of cuvettes corresponding to an individual measurement in the sequence of multiple measurements. In embodiments, the automated sample-handling system is further configured to move a second sample cuvette from one location to another. Both the first and second cuvettes can include a first magnetically active material, e.g. a metal that is attached to a top portion of both the first and second sample cuvettes. Here, the automated sample-handling system includes a second magnetically active material that magnetically attracts the first magnetically active material. For example, the second magnetically active material is an electromagnet, e.g. a computer-controlled electromagnet.

These and other advantages of the invention should be apparent from the following detailed description, and from the claims.

### BRIEF DESCRIPTION OF THE DRAWINGS

FIG. 1 is a schematic drawing of the SSMD according to the invention for automatically measuring coagulation parameters, blood parameters, and biomarkers and blood-based ions during a PCI procedure;

FIG. 2 is a schematic drawing of the SSMD of FIG. 1 featuring a measurement component with a collection of sensors;

FIG. 3 is a schematic drawing showing a collection of sample cuvettes processed and measured with the SSMD of FIG. 2;

FIG. 4 is a schematic drawing showing sensors within the measurement component of the SSMD and sample cuvettes before and after centrifugation;

FIGS. 5A-F are mechanical drawings of the SSMD of FIG. 1 at various stages of blood draw and measurement cycles;

FIGS. 6A and 6B show, respectively, schematic drawings of versions of a blood-extraction component used to automatically extract venous blood during and in between measurements;

FIG. 6C is a schematic drawing of a front surface of a mechanical component shown in FIGS. 6A and 6B for moving a mechanical sheath in and out of a patient's vein;

FIG. 7A is a side-view mechanical drawing of a measurement component of the SSMD featuring optical, electrical impedance, and mechanical sensors and a sample cuvette;

FIG. 7B is a photograph of the measurement component that was fabricated from the mechanical drawing in FIG. 7A;

FIG. 8A is a top-view mechanical drawing of the measurement component and sensors shown in FIG. 7A;

FIG. 8B is a photograph of the measurement component that was fabricated from the mechanical drawing in FIG. 8A;

FIG. 9 is a schematic drawing of the SSMD mounted on an IV pole and wirelessly communicating with a remote gateway device;

FIG. 10A is a photograph of the gateway device shown in FIG. 9;

FIG. 10B is a screen capture taken from a user interface running on the remote gateway device of FIG. 10A;

FIG. 11 is a wavelength-dependent optical absorption spectrum measured continuously over a wavelength range of λ=200-1000 nm with a conventional absorption spectrometer (from Thorlabs, Inc.), and also measured at discrete wavelengths over a similar wavelength range with a multi-wavelength optical spectrometer featuring a chip-level optical detector (from AMS, Inc.);

FIGS. 12A-12C show ACT values and plots of time-dependent optical waveforms measured with the multi-wavelength optical spectroscopy system of FIG. 2 from, respectively, whole blood taken from a patient before receiving heparin, whole blood taken from a patient after receiving heparin, and whole blood taken from a patient after receiving both heparin and protamine;

FIGS. 12D-12F are mathematical first derivatives of the time-dependent optical waveforms measured from, respectively, FIGS. 12A-12C;

FIGS. 13A-13F are plots of time-dependent optical waveforms measured with the multi-wavelength optical spectroscopy system of FIG. 2 from whole blood taken from a patient after receiving both heparin and protamine at, respectively, λ=415 nm. 2=480 nm, λ=515 nm, λ=555 nm, λ=630 nm, and λ=680 nm;

FIGS. 14A and 14B are photographs of whole blood mixed with a collection of reflective white beads taken using the imaging+mechanical system of FIG. 2 before and while activating the vibrator system, respectively;

FIG. 15A is a plot showing a time-dependent optical waveform measured with the multi-wavelength optical spectroscopy system of FIG. 2 and an explicit value of ACT measured with the imaging+mechanical system of FIG. 2;

FIG. 15B is a plot showing two time-dependent optical waveforms measured with the multi-wavelength optical spectroscopy system of FIG. 2 and predicted values of ACT estimated by processing early portions of the optical waveforms with a mathematical model;

FIG. 16A is a schematic drawing of an alternate embodiment of the measurement component featuring embodiments of the multi-wavelength optical spectroscopy system and the imaging+mechanical system that images the sample as it is rocked back and forth with a motor;

FIG. 16B shows a sample cuvette containing unclotted blood and used in the alternate embodiment of the measurement component of FIG. 16A before and after being rocked back and forth;

FIG. 16C shows a sample cuvette containing unclotted blood and used in the alternate embodiment of the measurement component of FIG. 16A before and after being rocked back and forth;

FIG. 17 is a photograph of a sample cuvette, taken by a digital camera within the measurement component, that shows whole blood separated into red/white blood cells and plasma after being centrifuged:

FIGS. 18A and 18B are scatter plots showing hematocrit values measured with a reference device and compared to, respectively, hematocrit values measured by the measurement component using images similar to those shown in FIG. 17 and hematocrit values measured with a standard POC device;

FIGS. 19A and 19B are scatter plots showing hemoglobin values measured with a reference device and compared to, respectively, hemoglobin values measured by the measurement component using both a Thorlabs spectrometer and the AMS AS7341, and hemoglobin values measured with a standard POC device;

FIG. 20A is an exploded mechanical drawing of a wearable sample cuvette according to an alternate embodiment of the invention that uses microfluidics to load blood samples into a measurement area;

FIG. 20B is a photograph of the wearable sample cuvette of FIG. 20A featuring sense and drive electrodes for making impedance/reactance measurements from human blood;

FIGS. 21A-21C are photographs of the sense and drive electrodes within the wearable sample cuvette of FIG. 20A sampling human blood and making measurements at different time periods to show increasing levels of coagulation;

FIG. 21D is a time-dependent plot of an electrical impedance waveform measured using the sense and drive electrodes of FIGS. 21A-21C; and

FIG. 22 is a flow chart showing steps used by a closed-loop system to measure ACT from a patient and, in response, deliver a dose of heparin to the patient.

### DETAILED DESCRIPTION OF THE INVENTION

### 1. Clinical Use Case

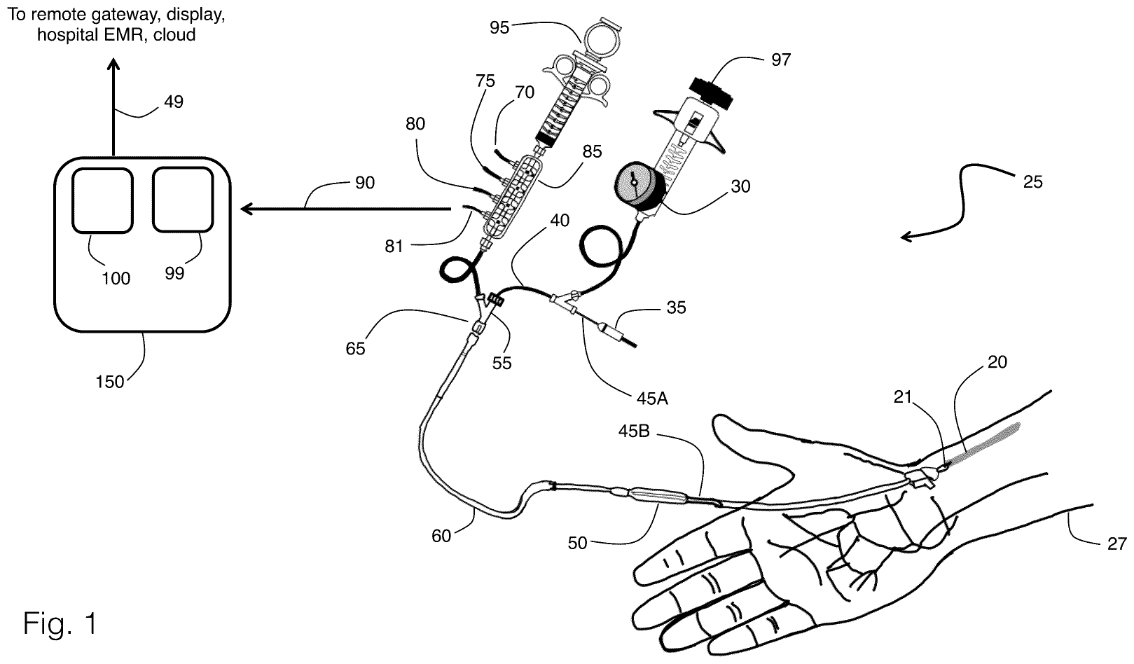

FIG. 1 shows equipment 25 used by a cardiologist during a conventional PCI procedure in a Cath Lab featuring an SSMD 150 that automatically extracts blood from a patient, and then measures coagulation parameters, blood parameters, biomarkers and blood-based ions according to the invention. More specifically, the SSMD 150 includes a blood-extraction component 99 that episodically removes small volumes (e.g. <1 cc) of blood from a patient, and a measurement component 100 that measures the removed blood to determine levels of the above-described compounds using a combination of optical, electrical, and mechanical techniques, described in detail below. Taken in combination, the blood-extraction 99 and measurement 100 components monitor multiple critical hematological parameters during interventional and surgical procedures (e.g. PCI) and post-surgery recovery in a manner that is quasi-continuous, real-time, and automated.

The SSMD 150 is particularly directed towards patients receiving heparin, a ubiquitous anti-coagulant used in procedures like PCI and TAVR to prevent blood clots. Heparin is typically dosed based on the patient's weight. Through the SSMD's quasi-continuous measurements of coagulation parameters—and particularly ACT and aPTT—clinicians can maintain a patient in a ‘therapeutic window’ and thus reduce the probability of deleterious clots and bleeds. The SSMD 150 potentially replaces standard POC devices and measurements conducted in a hospital's hematology lab; these episodic events require manual operation, can take hours to perform, and typically return values for just one parameter.

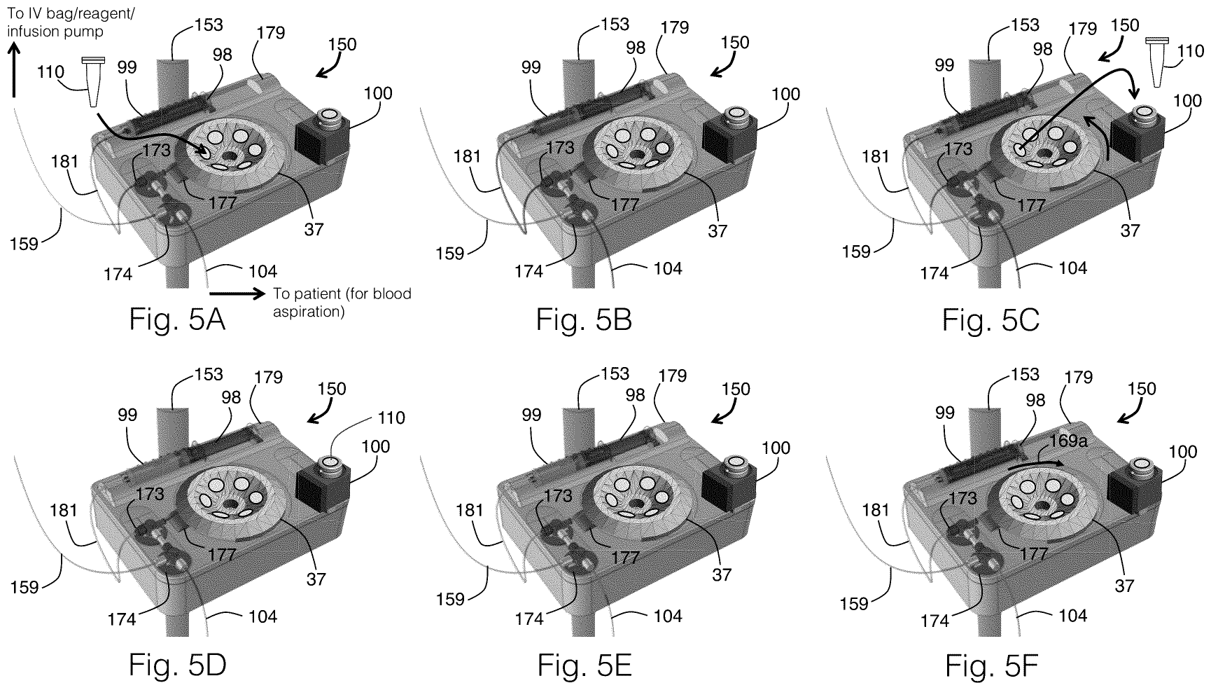

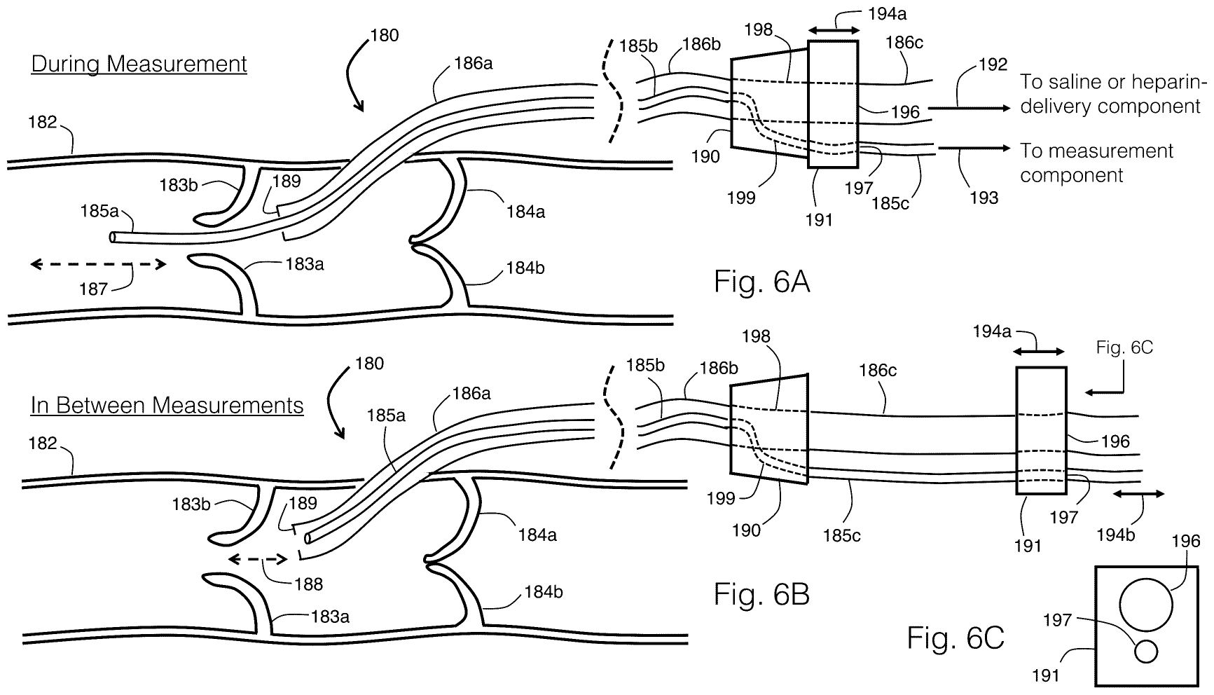

The blood-extraction component 99 in FIG. 1 features a pumping mechanism to aspirate blood (as shown in more detail in FIGS. 5A-5F, 6A, and 6B) that integrates with a port 81 disposed on a patient-connected manifold 85. In the embodiments shown in FIGS. 5A-5F, the pumping mechanism features a syringe 98 coupled to a linear actuator 179, effectively forming a computer-controlled ‘syringe pump’ that, during a measurement, automatically siphons off small volumes of blood from the patient. Referring back to FIG. 1, as indicated by the arrow 90, the blood-extraction component 99 removes small volumes of blood from the patient, and then automatically ports this to the measurement component 100, where, as described in detail below, it is then measured with the various optical, electrical, and mechanical sensors therein. Once a measurement is made, as indicated by arrow 49, wireless transmitters (typically Bluetooth®, Wi-Fi, or cellular radios; not shown in the figures) within the SSMD 150 transmit numerical values corresponding to these parameters, or time-dependent waveforms used to calculate them, to one of several different endpoints for further analysis, e.g. a remote gateway for display, a hospital's EMR system, or to a cloud-based software system.

During a PCI procedure, the SSMD 150 measures blood aspirated from the radial artery 20 of a patient 27. Alternatively, blood may be extracted from a vein, as described in more detail below. Blood in the artery is pressurized according to the patient's arterial blood pressure (e.g. systolic, diastolic, and mean pressures), and upon insertion of an in-dwelling catheter 21, blood flows from the artery 20 into a guiding catheter 60, and from there through an arterial sheath 65 to the manifold 85. Blood passes through the manifold 85 and port 81 into the SSMD's blood-extraction component 99. Other ports 70, 75, 80 within the manifold 85 can connect to other syringes or devices (not shown in the figure), e.g., to inject into a patient a contrast agent for imaging vessels during a procedure (e.g. port 80), inject saline or other compounds (e.g. port 75), or to dispose of waste (e.g. port 70). One of the ports may also connect to a pressure transducer and from there to a vital sign monitor (not shown in the figure) that, collectively, record time-dependent arterial waveforms indicating the patient's systolic, diastolic, and mean arterial blood pressures. The cardiologist can also use a syringe 95 to administer medication that flows through the manifold 85 and into the radial artery 20, and eventually into the patient 27.

The guiding catheter 60 additionally attaches to a Y-adapter 55, which helps to seal off and prevent fluid loss, and additionally connects to the arterial sheath 65. The arterial sheath 65 passes through the guiding catheter 60 and into the patient's radial artery 20. The cardiologist uses a coronary guidewire 45A, 45B to assist with advancing the guiding catheter 60 through the Y-adapter 55 and into the arterial sheath 65. A guiding system 35 controls the coronary guidewire 45A, 45B and guiding catheter 60. Once the arterial sheath 65 is inside the radial artery 20, the cardiologists uses the guiding system 35 to manipulate the guiding catheter 60 and advance a balloon catheter 40 connected to it towards the patient's heart. An inflation system 97 inflates the balloon catheter 40, and additionally connects to a pressure sensor 30 that measures the pressure within therein. During the procedure, the cardiologist uses the guiding system 35 to pass the balloon catheter 40 over the guiding catheter 60 to the artery identified as having a blockage. The cardiologist then uses the inflation system 97 to inflate a balloon 50 located at the tip of the balloon catheter 40. The inflating balloon 50 presses against plaque in the coronary artery to push it against a wall of the artery it is disposed in. The cardiologist may inflate the balloon 50 several times depending on the procedure. A stent (not shown in the figure) can be placed at the tip of the balloon catheter 40, over the balloon 50. In this case, during the procedure, the cardiologist uses the guiding system 35 to guide the balloon catheter 40 to the site of the blockage, and inflates the balloon 50 to expand the stent. After the stent is deployed within the artery, the cardiologist deflates the balloon 50 and, using the balloon catheter 40 and guiding catheter 60 controlled by the guiding system 35, removes the balloon 50 from the patient.

### 2. SSMD Measurement System

Throughout this procedure, the SSMD 150 aspirates blood and then measures the coagulation parameters, blood parameters, biomarkers and blood-based ions therein. To do this, the blood-extraction component 99 removes blood from the patient using a system similar to that shown in FIGS. 5A-5F, 6A, 6B, and then ports the blood sample to the measurement component 100, where measurements are then made as described in detail below.

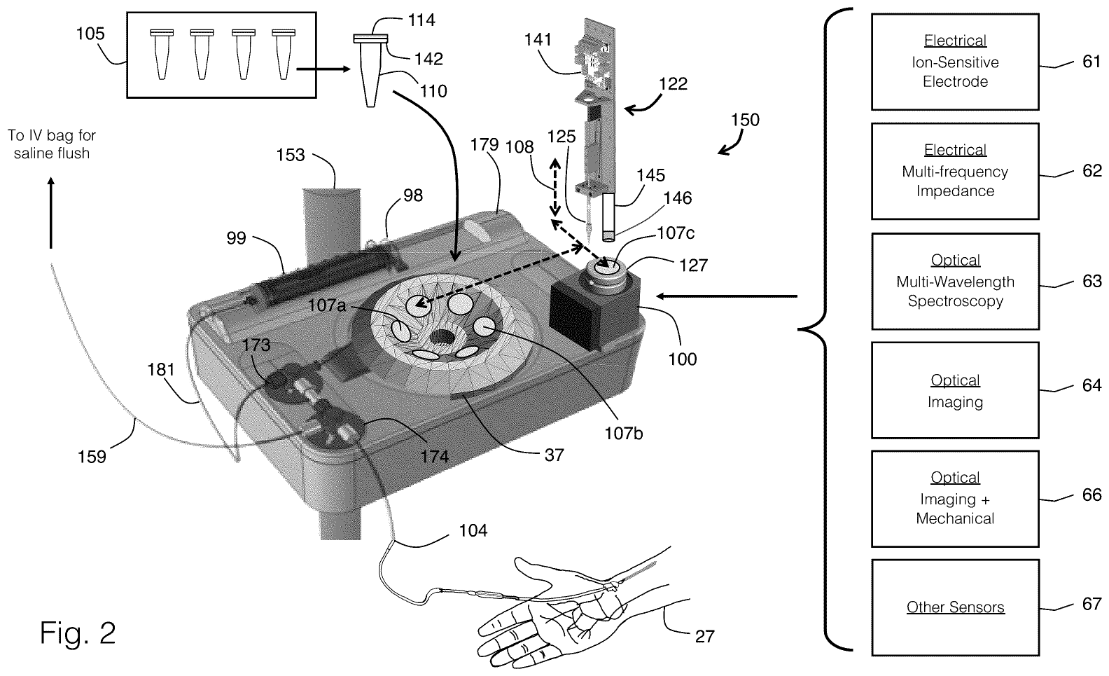

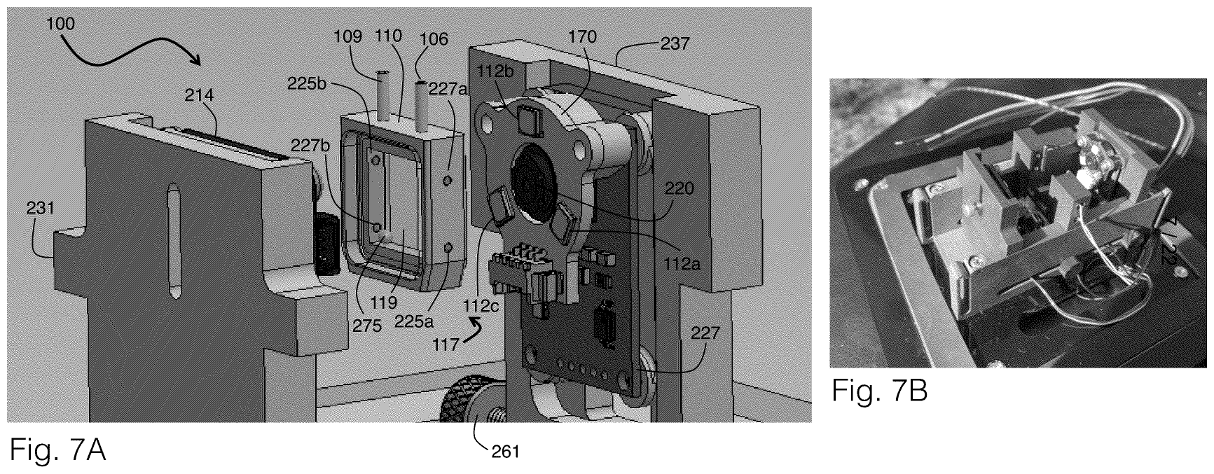

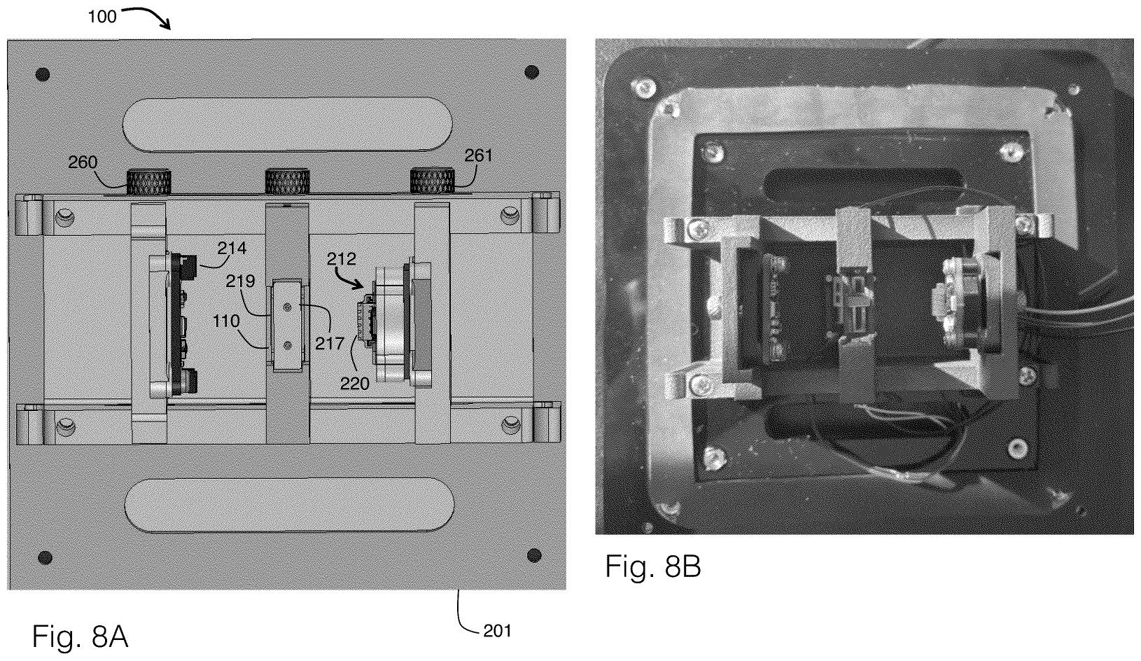

In embodiments, such as that shown in FIG. 2, the SSMD 150 includes a centrifuge 37 featuring a collection of openings 107a, 107b, each designed to receive a sample cuvette 110. Note that in FIG. 2 the cuvettes are shown to be cylindrical and feature curved faces, but in other embodiments the cuvettes may have a square or rectangular geometry and feature planar faces, as indicated in FIGS. 7 and 8. In preferred embodiments, the cuvettes are made from a transparent material, e.g. glass or plastic, that passes optical radiation ranging from the ultraviolet to the infrared. Each sample cuvette 110 features a cap portion 142 that includes a magnetically active metal 114. Also connected to the SSMD 150 is an automated robotic sample-handling system 122 featuring a cuvette-moving arm 145, terminated with an electromagnet 146 that is magnetically active only when a voltage is applied. A gantry system (not shown in the figure, but whose degrees of motion are indicated by the dashed lines 108) connected to the robotic sample-handling system 122 moves it along a three-dimensional axis.

During a measurement, the gantry system positions the automated robotic sample-handling system 122 so that the cuvette-moving arm 145 is disposed directly above a sample cuvette 110 within an array of cuvettes 105, and specifically above the magnetically active metal 114 disposed on its cap portion 142. A circuit board 141 within the cuvette-moving arm 145 supplies power to the electromagnet 146, which in turn attracts the magnetically active metal 114 capping the sample cuvette 110. This temporarily connects the sample cuvette 110 to the cuvette-moving arm 145, allowing it to be removed from the array 105. The gantry system moves the cuvette-moving arm 145 and the now-attached sample cuvette 110 above a first opening 107a in the centrifuge. The circuit board 141 then removes power from the electromagnet 146 to drop the sample cuvette 110 in the opening 107a. With the cuvette 110 in this position, the blood-extraction component 99 extracts blood from the patient 27 and fills the sample cuvette 110 according to the process described in more detail in FIGS. 5 and 6. Typically the cuvette-moving arm 145 sequentially loads multiple sample cuvettes 110 from the array 105 into the centrifuge 37, each in a different opening 107a, 107b.

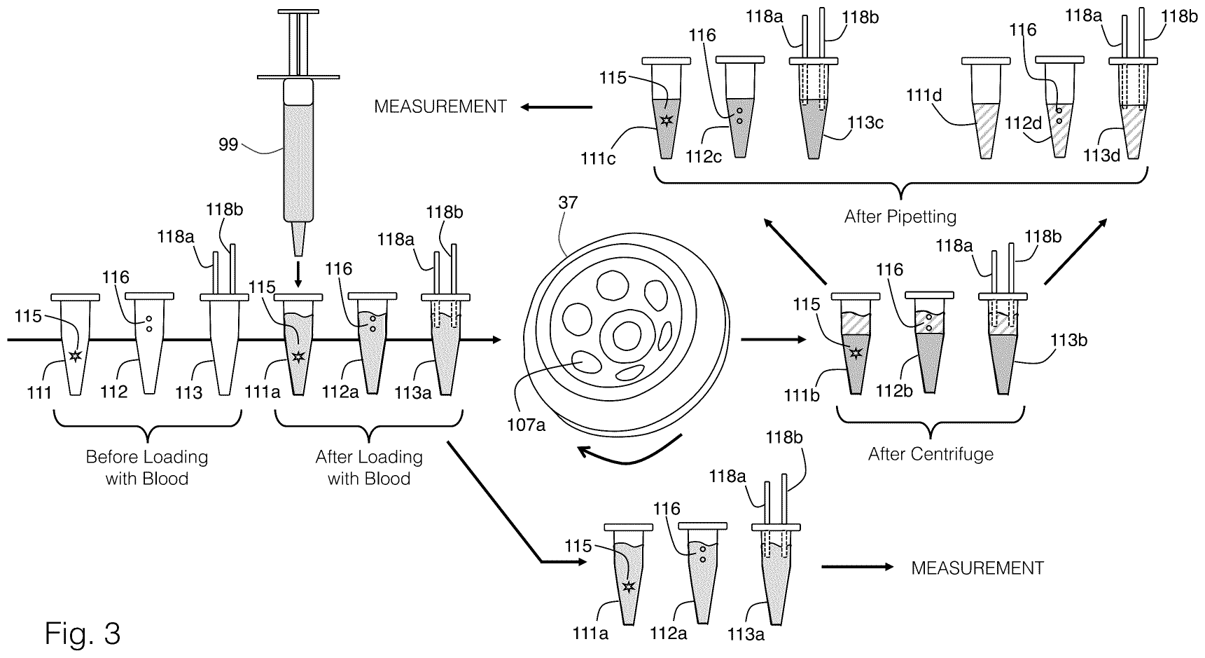

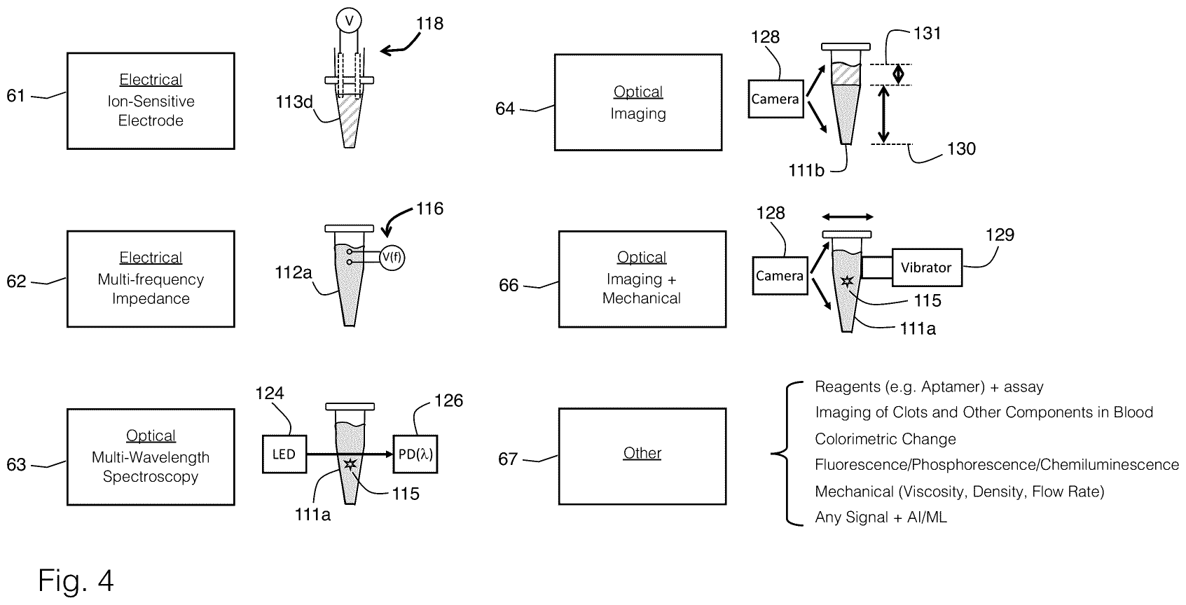

The SSMD 150 can measure multiple different properties from the patient's blood sample, as described in detail below, particularly with reference to FIGS. 3 and 4. Depending on the specific measurement, some blood samples require centrifugation to separate red blood cells (typically representing ˜45% of the total blood volume) from the ‘buffy coat’ that includes white blood cells and platelets (˜<1% of the total blood volume) and from plasma (˜55% of the total blood volume); if the blood is coagulated, centrifugation separates the blood cells from serum, which represents a percentage of the total blood volume similar to plasma. Other blood samples do not require centrifugation prior to measurements. If centrifugation is not required, the cuvette-moving arm 145 attaches to the sample cuvette 110 after the blood-extraction component 99 fills it with blood, as described with reference to FIG. 5, and moves it directly to the measurement component 100 within the SSMD 150.

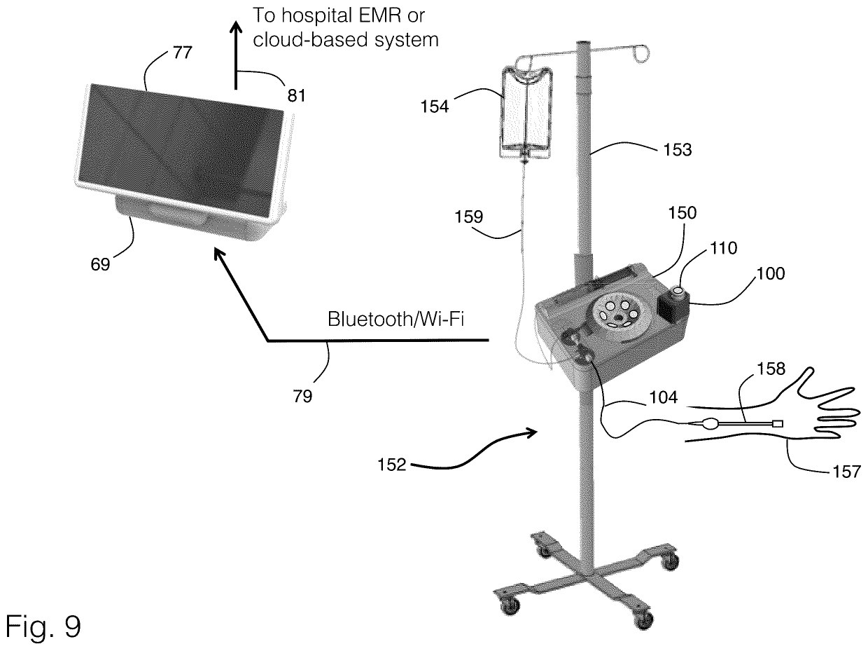

As described in more detail with reference to FIG. 5, the blood-extraction component 99 features a syringe 98 coupled to a computer-controlled linear actuator 179. Prior to any blood extraction, the cuvette-moving arm 145 moves from sample cuvette 110 from the array 105 to the opening 107a in the centrifuge 37. The linear actuator 179 then pushes fully forward a plunger within the syringe 98. A first segment of flexible tube 181 connects the syringe 98 to a first computer-controlled solenoid valve 173, which in turn features a fluid path to a sample head 177 proximal to the centrifuge 37 that deposits blood and saline (for flushing the system) into the sample cuvette 110 disposed in the opening 110a in the centrifuge 37. A second segment of flexible tube 159 connects a saline bag (not shown in the figure, but similar to component 154 in FIG. 9) which provides saline used to flush the system to a second computer-controlled solenoid valve 174. A third segment of flexible tube 104 connects to an in-dwelling catheter inserted in a patient (not shown in the figure, but similar to component 158 in FIG. 9). The entire SSMD can connect to an IV pole 153 disposed, e.g., proximal to the patient 27 in a Cath Lab or hospital bed.

Prior to a measurement, the cuvette-moving arm 145 presses down on the cap portion 142 of the sample cuvette 110, securing it so that no sample can leak out. For measurements not requiring centrifugation, the gantry system then moves the arm 145 and the non-centrifuged sample cuvette 110 to above an opening 107c above the measurement component 100, which includes a sample holder 127. Similar to the process described above, the circuit board 141 then removes power from the electromagnet 146 to drop the cuvette 110 in the opening 107c within the sample holder 127, where the measurement within the measurement component 100 commences. If centrifugation is required, the cuvette-moving arm 145 first deposits the sample cuvette 110 in the opening 107a in the centrifuge 37, and then the centrifuge 37 is powered and spun at high frequency (typically between 2000-6000 rpm, which can deliver centrifugal forces of up to 4000 g) for a predetermined period of time (typically between 2-15 minutes), thereby exerting a centripetal force that separates blood into its various components, e.g. red/white/platelet cells and plasma or serum. Typically, centrifugation time of at least 10 min and 1500 g is recommended for serum, and at least 15 min and 2000-3000 g for plasma. This action prepares the sample for specific measurements within the measurement component 100, as described in more detail with reference to FIGS. 3-4. After centrifugation, the gantry system delivers the sample cuvette 110 to the opening 107c within the sample holder 127, disposed above the measurement component 100, and the measurement commences, as described in more detail below.

For some measurements, it may be required to only include specific blood components in the sample cuvette 110, e.g. red blood cells, the buffy coat, plasma, or serum. To do this, the sample-handling system 122 includes a pipetting robot 125 that is moved about the sample cuvette 110 and inserted into the appropriate layer of blood after it is separated with centrifugation. Once the gantry system moves the pipetting robot 125 into place, the circuit board 141 translates an automated plunger (not shown in the figure) to extract a small volume into the pipette within the pipetting robot 125. The gantry system then moves the sample-handling system 122 to an empty sample cuvette within the array 105, pipettes the extracted sample component into it, moves this cuvette to the measurement component 100 so that the measurement may commence. When a measurement is complete, the cuvette-moving arm 145 and its distal electromagnet connect to the sample, and move it back to a portion of the array 105 dedicated to used cuvettes.

In some cases, the sample cuvette 110 may be preloaded with ethylenediaminetetraacetic acid (herein “EDTA”), which is a common anticoagulant used for most hematology procedures. Such an EDTA-loaded cuvette will preserve the blood sample for measurements conducted in the future. Addition of a clotting agent, as described in detail below, will counteract the effects of EDTA and activate the clotting process within the cuvette.

The measurement component 100 includes multiple sensor systems 61, 62, 63, 64, 66, 67, described in more detail below with reference to FIGS. 3-4, and also in the following co-pending patent application, the contents of which are hereby incorporated by reference: BLOOD-MEASURING SYSTEM—AUTOMATED METHODOLOGY FOR DETERMINING COAGULATION AND OTHER PARAMETERS, U.S.S.N XXXX, Filed YYYY. More specifically, and as indicated in FIG. 2, the measurement component includes one or more ion-specific electrodes 61 and a multi-frequency impedance system 62 for making electrical measurements, a multi-wavelength optical spectrometer 63, camera-based imaging system 64, and camera-coupled mechanical system 66 for making optical measurements, along with other sensors 67 described herein. Each of these systems is described in more detail below. The sample cuvette 110 may include specific components associated with a certain measurement, e.g. clotting agent for coagulation measurements, conductive sense and drive electrodes for impedance/reactance measurements, and conductive terminals to electrically connect with ion-specific electrodes for electrical measurements of specific ions. Each of these systems is described in more detail below.

FIG. 3, for example, shows schematic images of different versions of sample cuvettes 111, 112, 113 that the measurement component 100 uses for specific measurements. Sample cuvette 111, for example, includes a small volume of clotting agent 115—typically around 3-4 mg for a sample volume of blood of about 0.5 ml. Sample cuvette 112 includes metal contacts 116 that couple to spring-loaded pins in electrical contact with sense and drive electrodes within a multi-frequency impedance system in the measurement component 100. And sample cuvette 113 includes positive 118a and negative 118b terminals that connect to, respectively, a reference electrode and ion-specific electrode connected to an electrical system for measuring pH and blood-based ions in the blood sample, e.g. potassium, sodium, and calcium.

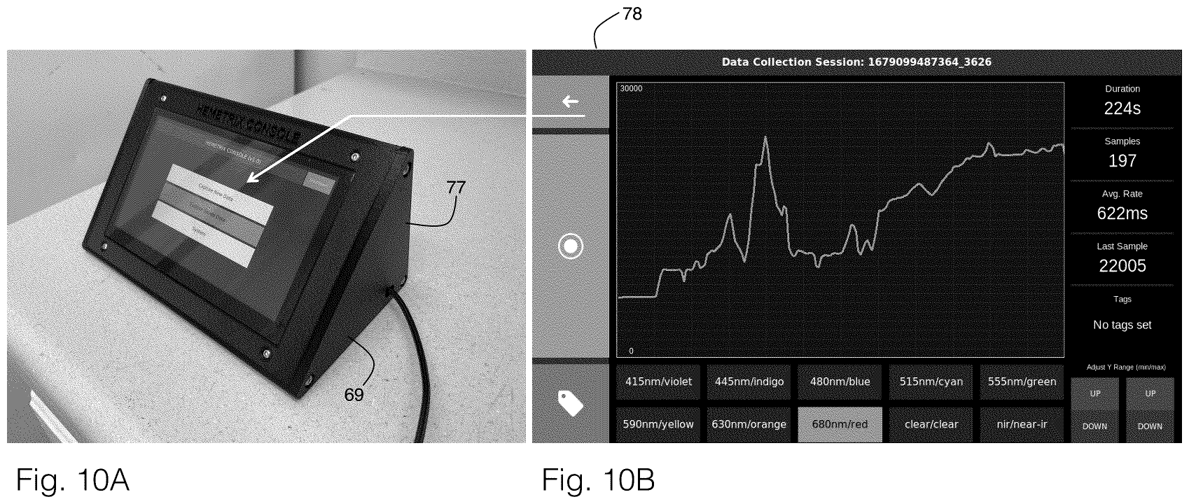

Prior to a measurement, the blood-extraction component extracts a blood sample from the patient; this process is fully automated and done according to a set schedule programmed into the SSMD using, e.g. a touchpanel display 77, such as that shown in FIGS. 9 and 10A, using an user interface similar to that shown in FIG. 10B. The blood-extraction component then loads blood into sample cuvettes 111, 112, 113, which the gantry system and robotic sample-handling system have placed into corresponding openings 107a in the centrifuge 37. This fills the sample cuvettes 111a, 112a, 113a with blood and readies them for measurement, either before or after centrifugation. For whole blood measurements, for example, the sample-handling system moves the filled sample cuvettes 111a, 112a, 113a into the measurement component, where measurements are then made.

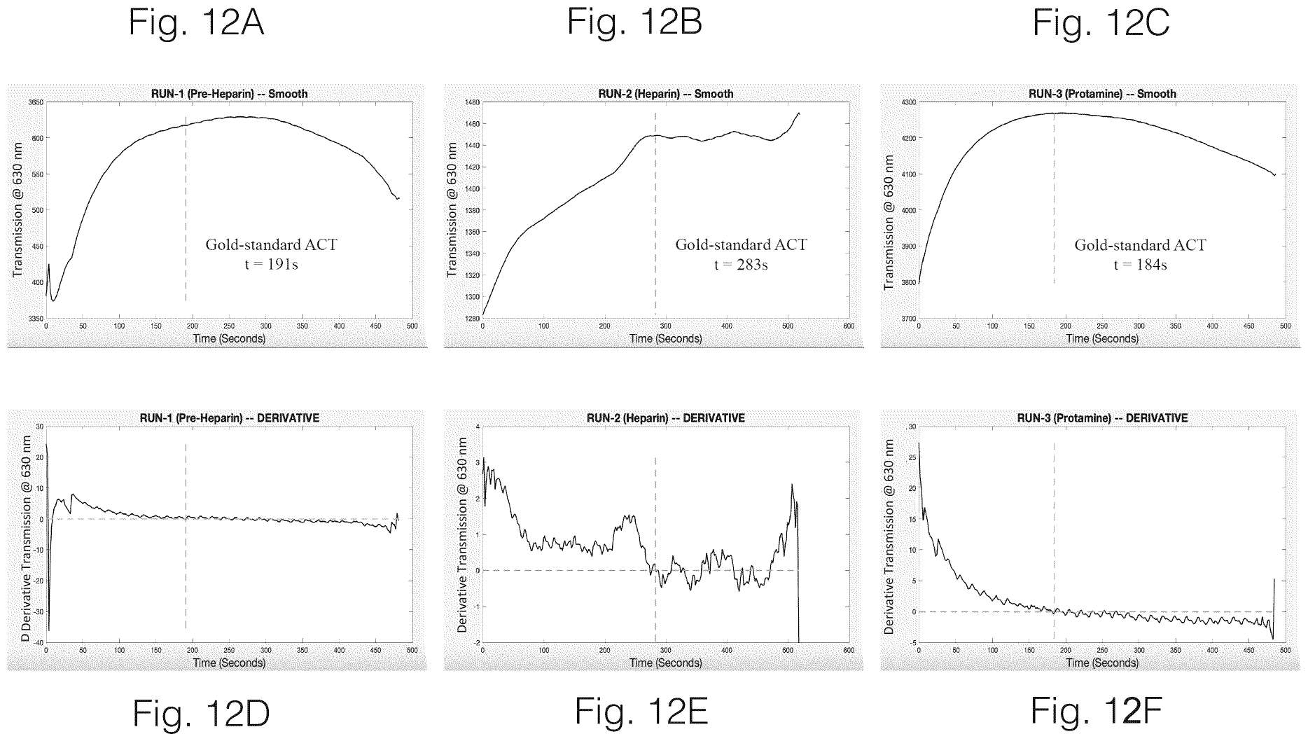

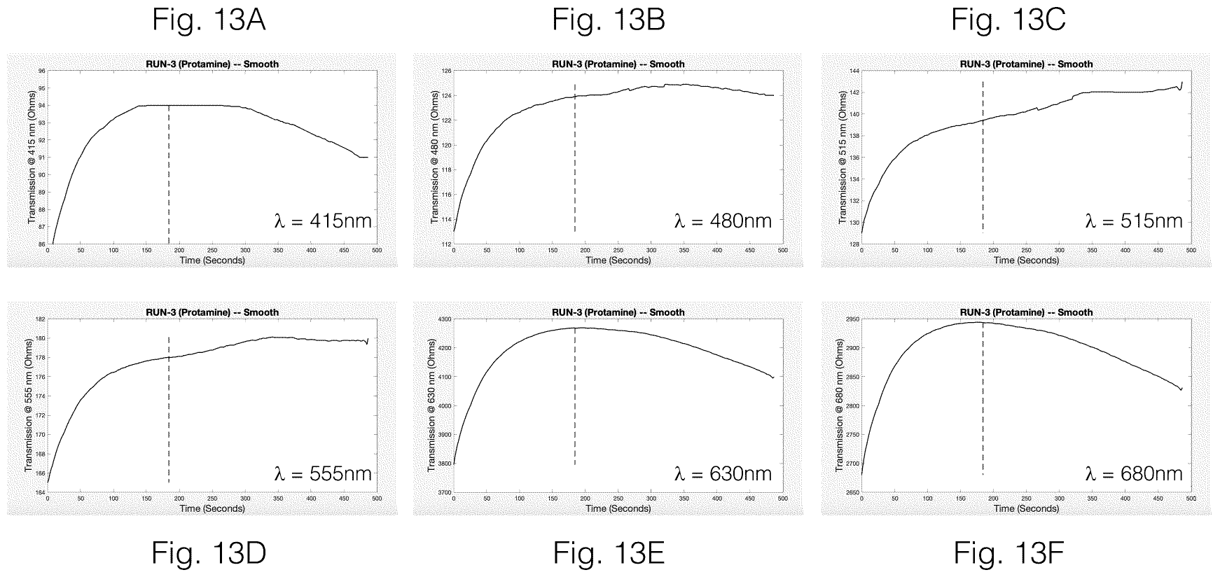

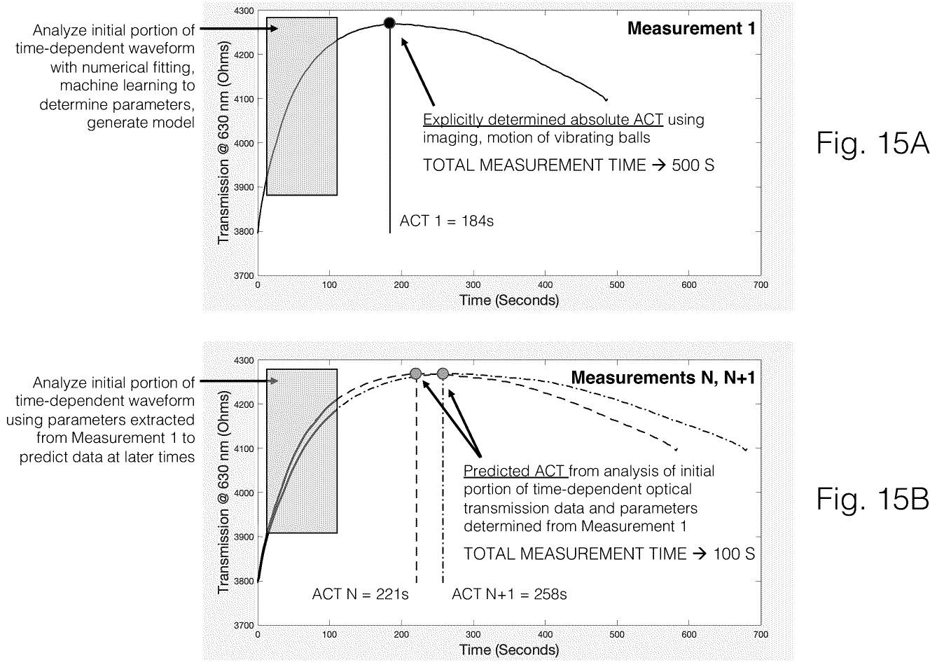

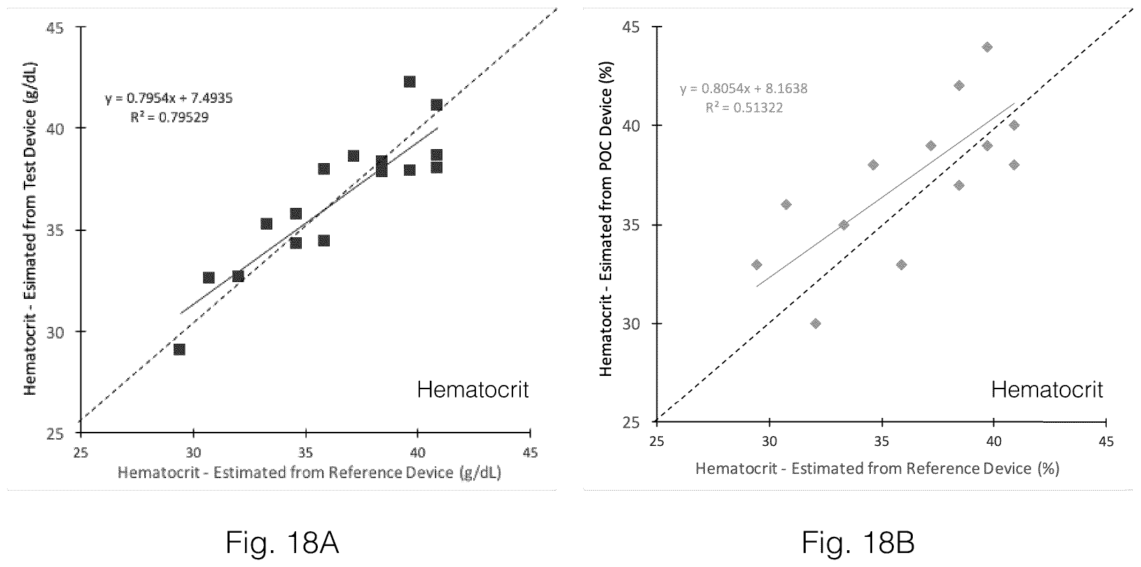

In one embodiment, the measurement component measures ACT from the sample cuvette 111a that includes the clotting agent 115, which is mixed with the whole blood prior to measurement. This measurement can be made with a range of different techniques, e.g. with multi-wavelength spectroscopy, as indicated above and shown in more detail by FIGS. 12-13, multi-frequency impedance, as indicated by FIG. 18, and/or a mechanical measurement coupled with optical imaging, as indicated by FIG. 15. A Normal range of ACT is 70-120 sec, with the therapeutic range for anticoagulation being 150-600 sec.

By making electrical contact with the metal contacts 116 in sample cuvette 112a, the multi-frequency impedance system within measurement component can measure electrical impedance and reactance at different frequencies of injected current in the whole blood sample. This, in turn, yields dielectric properties such as resistance and capacitance, which in turn can be used to characterize the sample's viscosity, water content, hematocrit, and the presence of certain biomarkers therein. Whole blood, a non-Newtonian fluid, has a typical viscosity of between 3.5-5.5 cP.

Similarly, circuitry in the measurement component associated with the ion-specific electrode can make electrical contact with the positive 118a and negative 118b terminals of the sample cuvette 113a to measure, among other things, pH of the whole blood sample. pH is a logarithmic measure of the hydrogen ion concentration in the whole blood sample, e.g. pH=−log [H+], where log is the base 10 logarithm and [H+] is the hydrogen ion concentration (typically with units of moles per liter). To measure pH, the negative terminal 118b serves as a pH-sensitive electrode that attracts positive H+ ions, and the positive terminal 118a serves as a reference electrode. The measurement component measures and digitizes the voltage between these electrodes, and then compares it to a pre-determined look-up table (typically determined beforehand with whole blood samples and a clinical study) to measure pH. In the absence of pathological states, the pH of the human body ranges between 7.35-7.45, with the average at 7.40.

Alternatively, other ion-specific electrodes can be used in place of the pH-sensitive electrode to measure certain ions. For example, a potassium-specific electrode can contact the negative terminal 118b, and a reference electrode contacts the positive terminal 118a, to measure potassium ions from the whole blood in the sample cuvette 113a. A normal potassium value in whole blood is 3.5-5.5 mEq/L; higher values may indicate, for example, hyperkalemia.

Prior to measurement, the sample-handling system may place sample cuvettes 111a, 112a, 113a into the centrifuge 37, where they are centrifuged to separate out plasma and serum—components that are some of the largest sources of biomarkers—from red/white/platelet blood cells. For example, FIG. 3 shows how the sample cuvette 111a, which contains whole blood and a small amount of clotting agent 115, can be centrifuged to yield sample cuvette 111b which contains serum, an amber-colored, protein-rich liquid that separates out when blood coagulates, and coagulated red blood cells. The pipetting robot shown in FIG. 2 can extract the serum from this sample and deposit it in a new sample cuvette 111d, which the measurement component then measures. Serum includes: i) all proteins not used in blood clotting; ii) all electrolytes, antibodies, antigens, hormones present in whole blood; and iii) any exogenous substances (e.g., drugs or microorganisms). For example, levels of albumin can be tested in a patient's serum. Here, using the pipetting robot 122, the reagent Bromcresol Purple is extracted from a reservoir in the array and mixed with serum in the sample cuvette 111d to form a complex. The multi-wavelength optical spectroscopy system in the measurement component monitors the change in absorbance at λ=600 nm from the resulting sample. The change in absorbance is directly proportional to the concentration of albumin in the sample. Other test with serum are also possible according to the invention.

In a related example, as indicated by sample cuvettes 112b and 113b, the centrifuge separates plasma and red/white blood cells from whole blood that has not been exposed to a clotting agent. The measurement component can then measure clotting aspects of blood directly from the plasma, which contains water (roughly 90%), fibrinogen and other clotting factors when separated from the red blood cells, ions, energy substrates, nutrients, metabolites, antibodies, proteins, and lipoproteins. For example, the prothrombin time (herein “PT”) test is performed by adding a source of tissue factor (e.g., a protein, thromboplastin, from homogenized brain tissue) to the plasma in the sample cuvette 112b to that converts prothrombin to thrombin. The mixture is then kept in a warm at 37° C. for one to two minutes, and clotting begins in the serum sample. The time required for the plasma to clot, as measure e.g. with optical, electrical, or imaging techniques as described herein, is the PT. PT measurements are typically conducted on patients on blood thinners other than heparin, e.g. warfarin; normal values of PT are typically 11-13.5 seconds. PT measurements are typically conducted using a parameter called the international normalized ratio (herein “INR”), which is a parameter used in a calculation based on results of a PT that is used to monitor individuals who are being treated with warfarin.

In related embodiments, testing for aPTT performed to investigate bleeding disorders and to monitor patients taking an anticlotting drug (e.g. heparin) which inhibits factors X and thrombin, while activating anti-thrombin. To test for aPTT, whole blood (absent of any clotting agent) is separated into red/white cells and plasma using the centrifuge 37. The pipetting robot then removes the plasma, and places it into another sample cuvette that also includes a clotting agent, such as Kaolin. Clotting is measured in the sample using optical, electrical, or imaging techniques as described herein to determine aPTT; a typical aPTT time is about 35 seconds.

In other embodiments, the reagent, for example, may be a chemical or biochemical compound that reacts with a compound in the blood. Examples of this include a reagent that features an aptamer designed to specifically bind to a clotting factor in the blood (e.g. a protein, such as thrombin). Preferably the aptamer is coupled to an optically measurable and/or labeling compound, such as a dye molecule that exhibits a colorimetric change. Aptamers are short sequences of artificial DNA, RNA, XNA, or peptide that bind a specific target molecule, or family of target molecules. They exhibit a range of affinities (KD in the pM to μM range) with little or no off-target binding and are sometimes classified as ‘chemical antibodies’. Aptamers (and, alternatively, antibodies) can be used in many of the same applications, but the nucleic acid-based structure of aptamers, which are mostly oligonucleotides, is very different from the amino acid-based structure of antibodies, which are proteins. This also means aptamers can be made quickly and at low cost with commercially available DNA sequencers.

Thrombin is an important component of the clotting cascade, and thus measuring it can be an effective way to characterize blood coagulation for a patient during surgery or recovery. Here, the aptamer is configured to bind to a fibrinogen binding site and is 5′-GGTTGGTGTGGTTGG-3. Or the parameter can be thrombin, and the aptamer is configured to bind to a heparin binding site and is 5′AGTCCGTGGTAGGGCAGGTTGGGGTGACT-3. Such an aptamer can be coupled to a labeling compound as described above, and then measured with the optical systems described herein to accurately detect the amount of thrombin in a blood sample.

For measurements that require adding a reagent such as an aptamer that binds to a specific protein to the whole blood sample, and then measuring the result sample (typically using an optical technique), the algorithm typically involves analyzing pre-determined, well-defined peaks in the optical and/or impedance spectra corresponding to the aptamer-protein compound, or an optically active compound bound to the aptamer, and then analyzing the resultant absorption spectrum as described above. In some cases, the optically active compound involved in this chemical reaction exhibits such a profound colorimetric change that a small-scale digital camera can detect it by collecting an image and using image-processing or pattern-recognition algorithms. Here, the image is processed with the algorithm, and the results of this analysis are compared to a look-up table featuring known concentrations of the sample to determine the concentration of the aptamer-bound protein. Other types of algorithms may also be used according to the invention.

Electrical methods, such as electrical contacts 116 that connect to impedance electrodes and positive 118a and negative 118b terminals that connect to, respectively ion-specific and reference electrodes, can also be used to test blood plasma, as indicated by sample cuvettes 112d and 113d. For example, positively charged ions, called “cations”, such as Na+, K+, Mg+, and Ca2+ are largely present in blood plasma, with Na+being the most prevalent and responsible for the plasma osmolarity. Such cations can be detected from sample cuvette 113d using the ion-specific electrodes described herein.

In related methods, red and white blood cells separated from plasma can be removed from centrifuged sample cuvettes 111b, 112b, 113b with the pipetting robot, and placed in new sample cuvettes 111c, 112c, and 113c for further testing. For example, counts of red blood cells, white blood cells, and platelets can be made using the camera system described herein, coupled with image processing. Techniques such as flow cytometry can also be incorporated in the measurement component, and used for this process. Hematocrit, which is the ratio of the volume of red blood cells to the total volume of blood, can be determined by imaging the centrifuged sample cuvette, such as sample cuvette 111b, 112b, 113b, and processing the image accordingly. A typical hematocrit value for men is in the range of 41-50%; normal levels for women are 36-48%

FIG. 4 shows specific details of how the above-mentioned tests are conducted on the sample cuvettes. Each measurement indicated in the figure is made within the measurement component, and can be conducted on any sample cuvette, e.g. one containing whole blood, one after centrifugation that features separated red/white/platelet blood cells and either plasma or serum in the same sample cuvette, or one that has one of the separated components pipetted out and dispersed in a new sample cuvette. As such, any of the sample cuvettes may also include reagents, clotting agents, or other compounds used for a specific blood test.

As indicated by the figure, the measurement component can include an ion-sensitive electrode sensor 61 that makes electrical contact with positive/negative electrode terminals 118 connected to the sample cuvette and the blood sample therein. In this case, the sample cuvette 113d includes plasma pipetted from the centrifuged whole blood sample. During a measurement, the positive/negative terminals 118 connect with ion-specific and reference electrodes in the ion-specific electrode sensor 61 to measure a voltage from the plasma within the sample cuvette. The voltage relates to the particular ion that is passed by the ion-sensitive electrode. It is digitized by an internal analog-to-digital converter within the measurement component, and compared by a computational module to a predetermined look-up table to estimate the concentration of the ion.

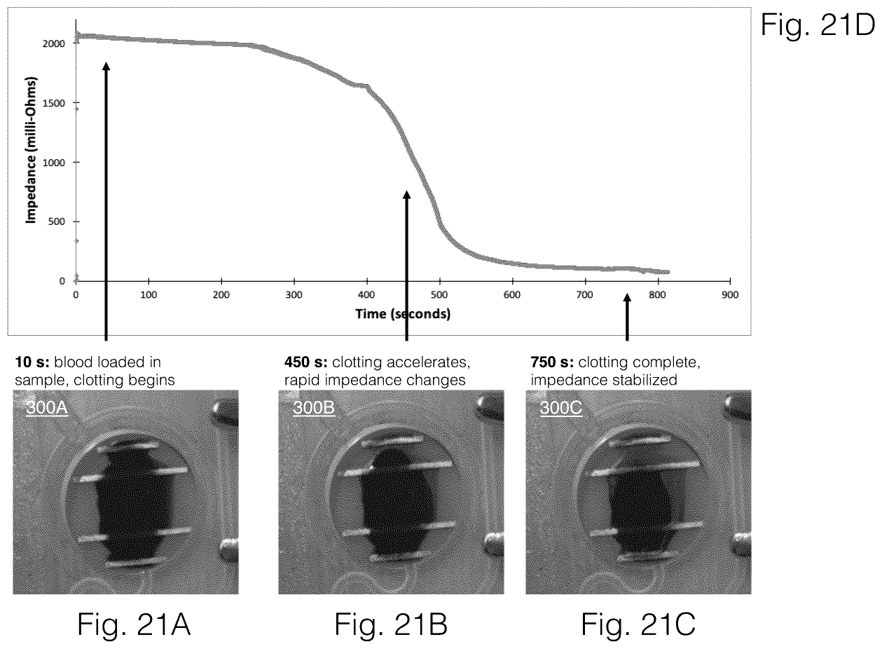

Multi-frequency impedance (and/or reactance) measurements are made in a similar manner when sense/drive electrodes associated with a multi-frequency impedance sensor 62 within the measurement component make an electrical connection with metal contacts 116 within the sample cuvette 112a, which as shown in the figure contains whole blood. The multi-frequency impedance sensor 62 is designed to measure impedance and reactance across the sample when the multi-frequency impedance sensor 62 injects electrical current at different drive frequencies into the sample cuvette 112a. The metal contacts measure bio-electric signals from the sample, which pass through the sense electrodes to the multi-frequency impedance sensor 62, which processes them to determine voltage at each frequency. Because the magnitude of the injected electrical current is controlled and known, the voltage relates to an impedance or reactance of the sample. The frequency dependence of these parameters can be analyzed to determine, e.g., a resonant frequency of the blood sample, which indicates its capacitance, or a Cole-Cole plot which indicates the dependence of impedance on reactance. Ultimately these parameters yield dielectric properties of the internal blood sample, which in turn can be used to detect biomarkers, clotting, and mechanical properties, such as viscosity. FIG. 21D, for example, shows a time-dependent impedance waveform measured from a blood sample.

The measurement component can also include a multi-wavelength optical spectrometer 63 that measures optical properties of blood within the sample cuvette 111a. The multi-wavelength optical spectrometer 63 typically includes an LED 124 that emits ‘white light’ radiation ranging from the ultraviolet (e.g. λ=350 nm) to the infrared (e.g. λ=700 nm). A broadband photodetector 126 (referred to as “PD(λ)” in the figure) detects the radiation after it passes through the sample cuvette 111a to determine the blood transmission spectrum or, conversely, absorption spectrum. Such spectra can then be used to determine properties of the blood, e.g. hemoglobin, cell count, presence of biomarkers, and clotting time. In FIG. 4, for example, the sample cuvette 111a includes a clotting agent 115, and the multi-wavelength optical spectrometer 63 measures time-dependent waveforms at different optical frequencies that indicate the blood clotting. FIG. 13, for example, shows these waveforms measured at different frequencies of visible light.

As a particular example, the multi-wavelength optical spectrometer 63 and its white light LED 124 and broadband photodetector 126 collectively measure hemoglobin from the sample cuvette 111a. Hemoglobin carries oxygen from the lungs to tissues and organs in the body, and then carries carbon dioxide back to the lungs. In this assay, no clotting agent 115 is present; in its place is “Drabkin's reagent”, a compound used for the quantitative, colorimetric determination of hemoglobin concentration in whole blood at 2=540 nm. Drabkin's reagent reacts with all forms of blood-based hemoglobin (except sulfhemoglobin, a pigment that normally occurs in only minute concentrations in blood). For this test, the sample cuvette 111a is preloaded with a small amount of Drabkin's reagent, and then filled with whole blood as described above. Hemoglobin within the blood mixes with the reagent, which converts all forms of hemoglobins to the colored protein cyanomethemoglobin, which is then measured at 2=540 nm with the LED 124 and photodetector 126. Software associated with the measurement component analyzes the peak at λ=540 nm, e.g. by determining its magnitude, area under a curve, or related parameter. The magnitude is then compared to a pre-determined look-up table to estimate the amount of hemoglobin in the blood sample. For men, a normal amount of hemoglobin is 13.8-17.2 g/dL; for females normal amounts are 12.1-15.1 g/dL.

In related embodiments, the multi-wavelength optical spectrometer 63 and its white light LED 124 and broadband photodetector 126 collectively measure creatinine from the sample cuvette 111a. Serum creatinine is a waste product formed by the spontaneous dehydration and breakdown of creatine, an amino acid derivative found in muscle tissue; levels of creatinine indicate kidney function. The rate of creatinine formation is fairly constant, with about 2 percent of creatine in the human body being converted to creatinine every 24 hours. Serum creatinine levels are elevated in patients with renal malfunction, especially in patients with decreased glomerular filtration. Measuring creatinine with the measurement component requires a reagent that was developed based on the Jaffe reaction. More specifically, blood within the sample cuvette is mixed with a clotting agent (e.g. Kaolin) to extract all clotting factors, and then centrifuged to separate out serum. The serum is then extracted with the pipetting robot, placed in a new sample cuvette, and then mixed with sodium borate to increase the pH to alkaline conditions (pH=13.1). The resulting sample is then mixed with a reagent containing picric acid, resulting in a colored complex that strongly absorbs optical radiation near λ=500 nm. The multi-wavelength optical spectrometer within the measurement component measures the resulting solution and processes an absorption peak in this area by digitizing the absorption spectrum, determining the peak amplitude at λ=500 nm, and comparing this value to a predetermined look-up table to estimate the amount of creatinine. Normal levels of creatinine are 0.7-1.3 mg/dL for men and 0.6-1.1 mg/dL for women.

In still other related embodiments, the multi-wavelength optical spectrometer 63 and its white light LED 124 and broadband photodetector 126 collectively measure glucose from the sample cuvette 111a. Generally, glucose measurements are based on interactions with one of three enzymes: hexokinase, glucose oxidase, or glucose-1-dehydrogenase. The hexokinase assay is the preferred reference method for measuring glucose with the multi-wavelength optical spectrometer 63, with assays using glucose oxidase and glucose-1-dehydrogenase typically deployed in low-cost, handheld monitors and wearable systems for continuous monitoring. For the hexokinase assay, glucose within either plasma or serum is converted to glucose-6-phosphate by hexokinase. The glucose-6-phosphate is then mixed with glucose-6-phosphate dehydrogenase in the presence of nicotinamide adenine dinucleotide (herein “NAD+”) to form nicotinamide adenine dinucleotide (herein “NADH”), with the resulting solution having strong optical absorption at λ=450 nm, which is proportional to the glucose concentration in the sample. Normal fasting glucose levels are typically between 70-100 mg/dL, with normal levels for non-fasting states being around 125 mg/dL.

In embodiments, the LED 124 in the multi-wavelength optical spectrometer 63 can be replaced with one or more coherent light sources, e.g. a laser. The photodetector 126 can be a single-IC solution with digitally programmable optical filters (e.g. the AMS AS7341 optical detector). Or, alternatively, the photodetector 126 can be replaced with a more traditional frequency-dependent detector, e.g. a CCD camera that detects optical frequencies dispersed with a diffraction grating or a prism.

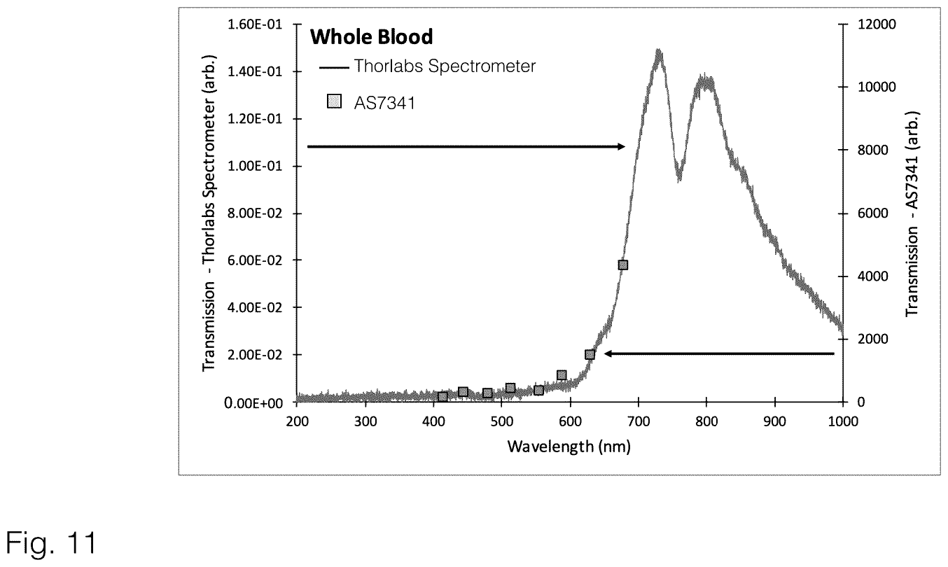

Alternatively, the photodetector described may be a spectral detector, such as the AS7262, AS7341, AS7343, or AS7421 chip-based solutions, all marketed by AMS Inc. (see, e.g., https://ams.com/en/spectral-sensing). These detectors are incorporated into a small-scale package (typically about 3×3 mm) and feature a sensitive photodiode positioned behind a set of miniaturized, computer-controlled Fabry-Perot etalons that act as programmable optical filters. During use, a computer program operating on a microcontroller within the SSMD and coupled to the AMS spectral detector sets a specific register, which in turn activates a specific optical filter that passes a narrow bandwidth of incident optical radiation (e.g., from λ=600-630 nm). The photodiode associated with the detector detects the narrow-band radiation in this region for a short period of time (e.g. a few milliseconds), generates a digital signal (e.g. a number of ‘counts’) that the microcontroller receives through a serial port. After this the computer program sets a new register that passes a new, narrow bandwidth of optical radiation (e.g., from λ=630-660 nm), and the process is repeated until a series of discrete signals spanning an optical spectrum is collected, with the series representing an optical absorption spectrum of the blood within the sample cuvette. The series of discrete signals serves as a proxy for a complete absorption spectrum, which, as described above, is typically measured with a much larger (and relatively expensive) apparatus, such as an optical absorption spectrometer featuring a tungsten light source, diffraction grating, and CCD camera. FIG. 11, for example, also shows such an absorption spectrum (continuous trace), as measured by an absorption spectrometer marketed by Thorlabs Inc. (https://www.thorlabs.com/newgrouppage9.cfm?objectgroup_id=3482). Discrete signals (individual squares) measured by the AS7341 part, which sequentially measures seven narrow bandwidths of optical radiation ranging from about λ=450-630 nm, each represented by an individual square in the plot. In FIG. 11 the two spectra are overlaid, indicating how the signals measured with the AS7341 part, while discrete, include comparable information to that determined with the Thorlabs spectrometer. The discrete data points measured by the AS7341 part can be ‘filled in’ using a computer algorithm, such as an interpolation or spline algorithm, to recreate the continuous spectrum measured with the conventional spectrometer.

Processing of the two-dimensional (or three-dimensional) trace determined from images like those shown in FIG. 12 may involve sophisticated algorithms, such those involving artificial intelligence (herein “AI”) and/or machine learning (herein “ML”), to extract and analyze information for this mechanical measurement. In one embodiment, once the time-dependent trace is extracted, it may be analyzed with numerical ‘fitting’ techniques that iteratively vary parameters of a ‘fitting function’ until the function best matches the time-dependent trace. Here, ‘best matching’ is typically determined with a fitting parameters called ‘χ²’ which represents a minimum error between the fitting function and the actual data.

In related embodiments, the time-dependent linear trace described above may be processed with a Fourier Transform, or similar mathematical operation, to transform the time-dependent trace into the frequency domain.

The measurement component can also include an imaging system 64, e.g. a digital camera 128 coupled with image processing software (e.g. software based on pattern recognition, AI, and/or ML). The digital camera 128 within the imaging system 64 takes high-resolution images of the sample cuvette 111b, which in the figure shows a post-centrifuge blood sample containing separated red/white/platelet cells and plasma. The high-resolution images show, for example, formation of blood clots, colorimetric changes brought on by the addition of chemical reagents, or other biomarkers or compounds that change color when mixed with blood components within the sample cuvette. In FIG. 4, the camera 128 within the imaging system 64 captures an image of the centrifuged sample cuvette 111b, which shows a first region 130 showing red/white/platelet cells (located at the bottom portion of the sample cuvette 111b), and a second region 131 showing plasma (located at the top portion of the sample cuvette 111b). Image processing software coupled with a computational system within the imaging system can collectively process the first 130 and second 131 regions to determine hematocrit, which is the ratio of the volume of red blood cells to the total volume of blood.

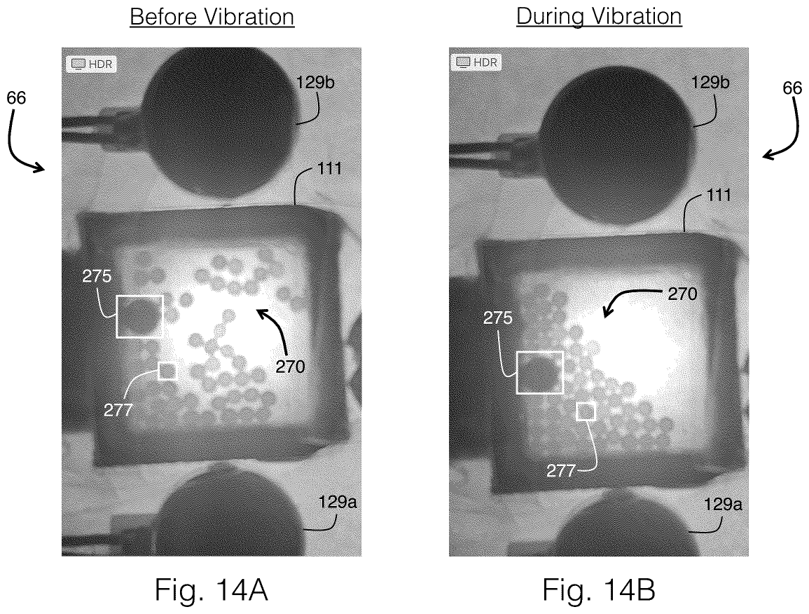

The measurement component can also include an imaging+mechanical system 66, which for example may include a digital camera 128 similar to that used in the imaging system 64, and a vibrator system 129 (similar, e.g., to a vibrating system in a mobile phone) that rapidly vibrates the sample cuvette 111a back and forth, thus causing blood and components therein to move accordingly. The sample cuvette 111a used here contains whole blood and a clotting agent 115. Additionally, it may include components that the imaging+mechanical system 66 can track as the vibrator system 129 vibrates the sample cuvette. FIG. 14 shows an example of this; here, the components are small, highly-reflective white beads and a relatively large metallic electromagnet. During a measurement, the vibrator system 129 rapidly vibrates the sample cuvette 111a, causing its contents (e.g., the reflective white beads in FIG. 14) to move back and forth, and eventually congregate in a corner of the cuvette, as specifically shown in FIG. 14B. As blood within the sample cuvette 111a clots, the ensemble movement of the white beads is gradually reduced, until full blood clotting within the cuvette 111a causes motion of the beads to completely halt. Tracking movement of the beads with the imaging+mechanical system 66, image-processing software, and image-analysis algorithms (e.g. ML and/or AI algorithms) can result in one or more time-dependent traces that, in turn, can be analyzed to determine clotting times (e.g. ACT for whole blood, PT or aPTT for plasma). The imaging+mechanical system 66 can also make other measurements with this technique, such as blood viscosity. Here, the viscosity can be determined, for example, by measuring one or more time-dependent traces using the above-described approach (reflective beads, vibrator system, imaging processing), and comparing features of the time-dependent traces with pre-determined values from a look-up table.

In embodiments, using the sensing components in FIG. 4, the measurement component can also be augmented to include other sensing components 67 that make additional measurements from blood within the sample cuvettes. These include: 1) assays featuring more complex reagents, e.g. aptamers, antibodies, proteins; 2) direct imaging of clot formation and other compounds in blood; 3) colorimetric change of blood within the sample cuvette, either occurring naturally or driven by addition of a reagent or similar chemical or biochemical compound; 4) emission of radiation via fluorescence, phosphorescence, or chemiluminescence, either taking place naturally, driven by a reagent, or induced by a light source, e.g. a laser or LED; 5) mechanical movements of natural or man-made compounds with the blood to determine mechanical properties such as viscosity, density, or flow rate; 6) measurements that detect acoustic properties of the sample; and 6) basically any signal generated and then processed with sophisticated computational techniques, such as AI or ML.

Based on the above, measurements of blood-based compounds made by the measurement component are summarized as follows:

| | | | | Sensor Used in |
|---|---|---|---|---|
| | | | | Measurement |
| Parameter | Blood Component | Centrifuge? | Reagent | Component |
| ACT | Whole blood (for | No | Kaolin, | Optical, imaging, |
| | patients receiving | | diatomaceous | imaging + |
| | heparin) | | earth | mechanical, multi- |
| | | | | frequency |
| | | | | impedance |
| aPTT | Plasma (for | Yes | Kaolin, | Optical, imaging, |
| | patients receiving | | diatomaceous | imaging + |
| | heparin) | | earth | mechanical, multi- |
| | | | | frequency |
| | | | | impedance |
| PT | Whole blood (for | No | Tissue factor | Optical, imaging, |
| | patients not | | (e.g., a protein, | imaging + |
| | receiving heparin) | | thromboplastin) | mechanical, multi- |
| | | | | frequency |
| | | | | impedance |
| Hemoglobin | Whole blood/red | Yes/No | Drabkin's | Optical, imaging |
| | blood cells | | reagent | |
| Hematocrit | Red/white/platelet | Yes | None | Imaging |
| | cells & plasma | | | |
| pH | Whole blood | No | None | Ion-sensitive |
| | | | | electrode |
| Creatinine | Serum | Yes | Sodium borate, | Optical |
| | | | Jaffe's reagent | |
| | | | (contains picric | |
| | | | acid) | |
| Glucose | Plasma, whole | Yes/No | Hexokinase, | Optical |
| | blood | | glucose-6- | |
| | | | phosphate | |
| | | | dehydrogenase, | |
| | | | NAD+ | |
| K+ (potassium) | Serum/plasma | Yes | None | Ion-sensitive |
| | | | | electrode |
| Na+ (sodium) | Serum/plasma | Yes | None | Ion-sensitive |
| | | | | electrode |
| Albumin | Serum | Yes | Bromcresol | Optical, imaging |
| | | | Purple | |
| Thrombin | Plasma | Yes | Specific aptamer | Optical, imaging |
| Lactate/lactic | Plasma | Yes | Specific aptamer | Optical, imaging |
| acid | | | | |
| BNP | Plasma | Yes | Specific aptamer | Optical, imaging |
| Cortisol | Plasma | Yes | Specific aptamer | Optical, imaging |
| Cell count | Red/white/platelet | Yes | None | Optical, imaging, |
| | cells | | | cytometry |

By including sets of sample cuvettes, the SSMD allows a collection of measurements to be made during a medical procedure, e.g. a PCI procedure. For example, prior to the procedure, a clinician may operate a user interface running on a touchpanel display (e.g. a touchpanel display 77 shown in FIGS. 9, 10A) to ‘program’ a measurement sequence into the SSMD. The measurement sequence may include the types of measurements the clinician desires to make during the procedure, along with how often they want to make the measurements (e.g. ACT measurements every 15 minutes, hemoglobin measurements every 60 minutes, potassium and chloride ion measurements made at the very beginning of the procedure, a PTT made at the very end of the procedure).

As with all data used in the hospital, a packet used to transmit the data between the SSMD and the touchpanel display is encrypted; the user interface operating on the display follows guidelines established by the Health Insurance Portability and Accountability Act of 1996 (herein “HIPPA”).

### 3. Examples of Measurements Made During Surgical Procedures and Post-Surgery Recovery

FIG. 9 shows a fully integrated measurement system 152 that features the SSMD 150 integrated with a conventional IV pole 153. The system 152 is used, for example, during a surgical procedure or post-surgery recovery. A catheter 158 inserted into a vein within an arm 157 of a patient aspirates blood, which passes through a first flexible tube 156 and into the SSMD 150. From there, a pipette (not shown in the figure) or a similar mechanism ports the blood into a sample cuvette 110 within a circular array 163 containing multiple sample cuvettes. Likewise, an IV bag 154 attached to the IV pole 153 provides fluids and/or reagents through a second flexible tube 159 and into the SSMD 150. A touchpanel display 77 features two-way communication with the SSMD 150, as indicated by the arrow 79. Communication can be through a wired mechanism (e.g. a serial cable), but is preferably made using a wireless protocol, e.g. Wi-Fi, Bluetooth®, or cellular. For example, and as described above, a customized user interface operates on the touchpanel display 77, allowing the clinician to program into the user interface a desired sequence of measurements to be made during a surgical procedure or post-surgery recovery. In alternate embodiments, the SSMD 150 can include functionality to make immediate, episodic measurements, as opposed to (or in addition to) pre-programmed periodic measurements. Here, the user interface operating on the touchpanel display 77 includes a software ‘on-demand button’ that, once clicked by the clinician, sends a code through the wireless interface to the SSMD 150, instructing it to immediately make a measurement. This can occur within seconds, after which the corresponding numerical value is transmitted back to the touchpanel display 77 for display to the clinician.

Measurements made in this manner during the surgical procedure typically focus on ACT, hemoglobin, and hematocrit, as these are standard for patients receiving heparin, and typically occur at relatively high frequency (e.g. every 15-30 minutes) using POC devices. Conversely, measurements made during post-surgical recovery typically focus on PTT, hemoglobin, and other blood-based compounds, such as glucose, potassium and chloride ions, lactate and lactic acid, cortisol, and other biomarkers. These measurements, which are typically made from patients on heparin drips, usually occur at relatively low frequencies (e.g. every few hours) using blood tests in the hospital's hematology laboratory. In both cases, the SSMD 150 makes the measurements automatically without requiring any human interaction. Numerical values corresponding to measurements made by the SSMD 150 are transmitted through Wi-Fi or Bluetooth® (again, according to the arrow 79) back to the touchpanel display 77, where they are displayed to the clinician and additionally transmitted to the hospital EMR or similar cloud-based system, as indicated by arrow 81. Note that the touchpanel display 77 is typically connected to a computer 69 configured to process information that the SSMD 150 generates. For example, the computer 69 can simply plot out graphical representations of the data for the clinician, e.g. trends, histograms, or other charts that indicate the patient's status. More sophisticated operations performed by the computer 69 include operating algorithms that process data from the SSMD 150 in various ways. For example, the algorithms may analyze trends in measurements made by the SSMD to predict values of future measurements before they are actually made. Or they may collectively analyze multiple parameters measured by the SSMD (e.g. both ACT and hemoglobin) to predict a physiological state corresponding to the patient.

As a particular example, during a PCI procedure, the fully integrated measurement system 152 featuring the SSMD 150 is connected to a patient in the Cath Lab. The surgeon performing the procedure typically requires ACT values every 30 minutes or so; it is particularly important that measurements are made each time the surgeon makes certain interactions with the patient, e.g. places a balloon catheter or stent, as described above with reference to FIG. 1. With the SSMD 150, the surgeon can focus on the procedure at hand and simply view the touchpanel display 77 to get ACT values that are measurement periodically or, as described, on demand. In some embodiments, as described below with reference to FIG. 15, ACT values are measured in a continuous manner using a predictive algorithm. Here, the numerical values representing these parameters are continuously streamed to the touchpanel display 77 for the surgeon to view.

During post-surgery recovery, the fully integrated measurement system 152 and SSMD 150 can potentially ameliorate life-threatening conditions such as sepsis and internal bleeds which are indicated by, respectively, lactate, lactic acid, hemoglobin, and hematocrit values corresponding to the patient. Typically, these values are measured in the hospital's hematology laboratory, a process that can take hours; the SSMD 150 can make comparable measurements in a matter of minutes. For sepsis, lactate is a chemical naturally produced by the body to fuel the cells during times of stress. Its presence in elevated quantities is commonly associated with sepsis and severe inflammatory response syndrome. When blood is lost, the body quickly pulls water from tissues outside the bloodstream in an attempt to keep the blood vessels filled. As a result, the blood is diluted, and the hematocrit (the percentage of red blood cells in the total amount of blood in the body, or blood volume) is reduced. In these and other cases, the SSMD 150 can detect the appropriate parameter, forward it to the hospital EMR, where a clinician can then view it to diagnose the patient.

The measurement system 152 and SSMD 150 can also characterize a patient's fluid state, e.g. whether they are hypovolemic (a decreased volume of circulating blood), hypervolemic (a state of fluid overload), or normovolemic (normal blood volume). Hypovolemic shock, in particular, is a potentially life-threatening condition where early recognition and appropriate management are essential. Hypovolemic shock is circulatory failure due to effective intravascular volume loss (fluids or blood), which in turn leads to tissue hypoperfusion and tissue hypoxia. If left untreated, hypovolemic shock can lead to ischemic injury of vital organs, leading to multiorgan failure. The measurement system 152 can characterize various values that can be abnormal in hypovolemic shock. Patients can have increased serum creatinine due to prerenal kidney failure. Also, hyperkalemia or hypokalemia. Lactic acidosis can be present as a result of anaerobic metabolism. In cases of hemorrhagic shock, hematocrit and hemoglobin can be critically low. However, with a reduction in relative plasma volume, hematocrit and hemoglobin can be increased due to hemoconcentration. Once the measurements system 152 detects these conditions, early recognition and treatment with volume resuscitation to restore normovolemia can be life-saving.

FIGS. 5A-SF show more specifically how an SSMD 150 attached to an IV pole 153 performs the following. 1) aspirates blood from the patient with the blood-extraction component 99; 2) loads blood from the patient into a sample cuvette 110 which has been placed in a centrifuge 37; 3) (for certain measurements) activates the centrifuge 37 to spin down the blood sample, thereby separating red/white/platelet cells from plasma; 4) makes a measurement of blood within the sample cuvette 110 with the measurement component 100; 5) flushes the system with saline to remove any residual blood from the system, and 6) then repeats the process with a new blood sample.

Referring first to FIG. 5A, the SSMD 150 shown in this figure is about to start a measurement. Here, the blood-extraction component 99 features a syringe 98 coupled to a computer-controlled linear actuator 179. Prior to any blood extraction, a plunger within the syringe 98 is pushed fully forward. A first segment of flexible tube 181 connects the syringe 98 to a first computer-controlled solenoid valve 173, which in turn features a fluid path to a measurement head 177 that deposits blood into the sample cuvette 110. The sample-handling system (component 122 in FIG. 2) moves the sample cuvette 110 from the array of cells (component 105 in FIG. 2) into an opening in the centrifuge 37, where it can be filled with blood. A second segment of flexible tube 159 connects a saline bag (not shown in the figure, but similar to component 154 in FIG. 9) which provides saline to a second computer-controlled solenoid valve 174. A third segment of flexible tube 104 connects to an in-dwelling catheter inserted in a patient (not shown in the figure, but similar to component 158 in FIG. 9).

FIG. 5B shows a measurement upon initiation. Computer code operating on the SSMD 150 activates the linear actuator 179, which in turn pulls back the plunger on the syringe 98. The computer code also moves both the first 173 and second 174 solenoid valves such that suction from the syringe 98 aspirates blood from the patient, through the third segment of flexible tube 104, and finally into the first segment of flexible tube 181, where it is held for a short period of time.

FIG. 5C shows how blood aspirated from the patient flows into the sample cuvette 110, which is loaded in the centrifuge 37. Once the blood-extraction system 99 deposits blood in the first segment of flexible tube 181, computer code again moves both the first 173 and second 174 solenoid valves. The computer code also moves the linear actuator 179 forward, thus depressing the plunger on the syringe 98 and pushing blood from the first segment of flexible tube 181 past the first solenoid valve 173, and into the measurement head 177, which then delivers it to the sample cuvette 110 positioned within the centrifuge 37. For purposes of example, in this particular measurement the SSMD measures ACT, and thus the sample cuvette includes a small amount of clotting agent. Once blood is deposited in the sample cuvette 110, the centrifuge briefly spins to mix the blood and clotting agent but not separate blood cells from plasma. This mixes the contents within the sample cuvette, after which the sample-handling system moves the sample cuvette 110 from the centrifuge 37 into the measurement system 100, where ACT is measured as described herein.

FIG. 5D shows how saline is used to flush the segments of flexible tube in preparation for a follow-on measurement. Here, once again, computer code adjusts the first 173 and second 174 solenoid valve and the linear actuator 179, the latter of which pulls back the syringe's plunger to draw saline from an IV bag (again, similar to the IV bag 154 shown in FIG. 9, but not shown in this figure) through the second segment of flexible tube 159, second solenoid valve 174, first solenoid valve 173, and finally into the first segment of flexible tube 181 where, like the blood shown in FIG. 5B, it sits for a brief moment. FIG. 5E shows how, at this point, computer code moves the first solenoid valve 173 and linear actuator 179, which pushes the syringe's plunger a partial distance to drive saline from the first segment of flexible tube 181, past the first solenoid valve 173 and measurement head 177. This flushes all lines not connected to the patient of all residual blood. Then, as shown in FIG. 5F, computer code moves the first 173 and second 174 solenoid valves, and drives fully forward the syringe's plunger to push a volume of saline through the first 173 and second 174 solenoid valves, through the third segment of flexible tube 104 and back into the patient. This clears the remaining line (the third segment of flexible tube 104) of any residual fluids. The sample-handling system then removes the previous sample cuvette, and positions a new sample cuvette underneath the measurement head 177, and a new measurement then commences, as described above.

In embodiments, the blood-extraction component 99 shown in FIGS. 5A-F connects to a manifold (e.g. component 85 in FIG. 1), which in turn connects to the patient's arterial system. Here, the blood that the blood-extraction component extracts is fully oxygenated arterial blood that the measurement component then tests as described above. Alternatively, the blood-extraction component couples to the patient's venous system, and extracts partially oxygenated venous blood that is then sent to the measurement component for testing. An assumption here is that the degree of oxygenation in the blood has little to no impact on its coagulation, biomarkers, or ions therein. In this embodiment, the blood-extraction component typically includes a venous catheter that is inserted into the patient's vein prior to measurement, e.g. before the PCI procedure. The venous catheter couples to a syringe (e.g. component 98 in FIG. 1) and computer-controlled linear actuator (component 179 in FIG. 1), which then draws a blood sample in a manner similar to that shown in FIGS. 5A-F. In related embodiments, such as that shown in FIGS. 6A-6C and described in detail below, the blood-extraction component includes other mechanisms for drawing blood, e.g. a computer-controlled retractable sheath that periodically inserts into the catheter and is disposed past valves in the vein so that it can better sample flowing blood. Here, the retractable sheath couples to the syringe, which extracts blood when moved by the linear actuator. The sheath is then removed once blood is drawn.

Periodically inserting and extracting the sheath through this mechanism avoids clotting near the sheath's opening, which can occur if it is left in the vein for too long. Additionally, clotting can be further reduced by inserting the sheath past a valve in the patient's venous system that is proximal to the insertion site of the catheter. Here, ‘fresh’ blood also flows at a relatively high rate, and is ideal for extracting prior to delivery to the measurement component.

Referring now to FIGS. 6A-6C, another embodiment of the blood-extraction system features a specialized catheter 180 inserted in a patient's vein 182. During measurements, the catheter 180 automatically draws blood and transfers it to the measurement component described above (FIG. 6A), and then between measurements withdraws (FIG. 6B) to avoid clotting and hemolysis near the catheter's tip 189. The catheter 180 features a flexible IV tube 186a, 186b, 186c which encloses a retractable sheath 185a, 185b, 185c. Middle portions of the flexible IV tube 186b and retractable sheath 185b connect to a front face of a manifold/fitting 190 that includes internal channels 198, 199 that effectively separate the coupled, co-centric portions of the flexible IV tube 186b and retractable sheath 185b. Once separated, distal portions 185c, 186c of these components disposed outside the vein 182 exit a back face of the manifold/fitting 190. A movable mount 191 attaches to a computer-controlled linear actuator (not shown in the figure) that translates back and forth, as indicated by arrow 194a, 194b. The distal, out-of-vein portion of the retractable sheath 185c passes through a relatively small opening 197 in the mount 191, which secures it so that both the retractable sheath 185c and movable mount 191 traverse in concert, as indicated by arrows 194a, 194b. Conversely, the distal, out-of-vein portion of the flexible IV tube 186c passes through a relatively large opening 196 in the mount 191 which does not secure it in any way, but instead slides along its outer surface when the computer-controlled linear actuator pulls the mount 191 away from the manifold/fitting 190; this means the flexible IV tube does not move in-between measurements. An end portion of the distal, out-of-vein portion of the flexible IV tube 186c ultimately connects to an infusion system (e.g. a pump) that delivers, e.g., saline for flushing the line and/or heparin to the patient; this is indicated by arrow 192. The mated distal, out-of-vein portion of the retractable sheath 185c connects directly to the measurement component, as indicated by arrow 193. With this configuration, the computer-controlled linear actuator can move the mount 191 away from the manifold/fitting 190 and retract all portions of the retractable sheath 185a, 185b, 185c, as indicated by arrow 194b, while keeping all portions of the flexible IV tube 186a, 186b, 186c in place within the vein 182.

To measure coagulation parameters, blood parameters, biomarkers and blood-based ions from a patient's blood, a clinician inserts the specialized catheter 180 into the patient's vein 182, as shown in FIGS. 6A and 6B. The vein 182, for example, may be the patient's radial vein, or any other vein located on their body, and preferably located in an easily accessible location, e.g. their arm or hand, with adequate blood flow. As shown in the figures, the catheter can be inserted so that its tip 189 is located between a first valve with open flaps 183a, 183b, and a second valve with closed flaps 184a, 184b. (Note: for simplicity, in both these figures the first valve is shown as temporarily open, and the second valve as temporarily closed; for an actual patient, these valves will open and close periodically to move blood within the vein.) During a measurement, as shown in FIG. 6A, the computer-controlled linear actuator pushes the movable mount 191 up against the manifold/fitting 190, thereby moving the portion of the retractable sheath 185a past the tip 189 of the distal portion of flexible IV tubing 186a and the open flaps 183a, 183b of the first valve. This action moves the distal portion of the retractable sheath 185a into a region where blood tends to flow a higher velocity, as indicated by arrow 187, as compared to blood near the tip 189 where blood flows at a relatively lower velocity, as indicated by arrow 188. The blood flowing at a relatively high velocity is freely circulating and lacks any small clots and/or cells impacted by hemolysis that tend to congregate around the tip 189; it is considered fresh and better for testing coagulation parameters, blood parameters, biomarkers and blood-based ions. A pump (not shown in the figure) connected to the distal, out-of-the-vein portion of the retractable sheath 185c draws out a small volume of blood, typically between 0.1-1.0 ml over a period of a few seconds, which passes to the measurement component for testing, as indicated by arrow 193.

When the measurement is complete, the computer-controlled linear actuator pulls the mount 191 away from the manifold/fitting 190, thereby pulling the entire retractable sheath, and most importantly, its distal in-vein portion 185a past the open flaps 183a, 183b of the first valve, and past the tip 189, where it remains until a new measurement initiates. This effectively shields it from blood and, when coupled with heparin flowing through the distal portion of the flexible IV tube 186a, prevents small clots from blocking flow within the retractable sheath 185a, 185b, 185c The above-described process is then repeated for each new measurement.

In other embodiments related to FIGS. 6A-6C, the linear actuator that translates the retractable sheath can be replaced with other mechanisms. For example, a mechanism based on one or more rotating gears, e.g. ‘rollers’ that are rotated by a computer-controlled stepper motor, can include ‘teeth’ that grip the retractable sheath shown in these figures and pull it in and out of the flexible IV tube to achieve the same results as shown above. Other similar mechanical mechanisms can be used for this purpose.

### 4. Physical Embodiments of the SSMD

FIGS. 7A and 7B show a side view of, respectively, a mechanical drawing and photograph of the sample cuvette 110 and measurement component 100 used within the SSMD. FIGS. 8A and 8B show a top view and photograph of, respectively, these same components. During a measurement, the sample cuvette 110 receives blood (and other reagents) through an input port 109 from the blood-extraction component, as described with reference to FIG. 2. The sample cuvette 110 also includes a venting port 106 that vents air displaced from the sample cuvette 110 to the ambient environment. The sample cuvette 110 encloses a metal ball 275 that is magnetically active, and features optically transparent windows 117, 119 on both faces. The sides of the sample cuvette 110, near its top and bottom portions, feature pressed-in metal pins 225a, 225b, 227a, 227b functioning as sense (225a, 225b) and drive (227a, 227b) electrodes for the electrical impedance/reactance measurements described above. During a measurement, the cuvette-moving arm moves the sample cuvette 110 into a mated slot (not shown in the figure) that includes spring-loaded metal contacts (e.g. pogo pins) that press against the metal pins 225a, 225b, 227a, 227b and connect them to appropriate sense/drive terminals in an underlying circuit.

Alternatively, in embodiments, the transparent windows 117, 119 are coated with ITO electrodes, which as described above and both electrically conductive and optically transparent.

In one embodiment, to measure ACT, a magnet attached to a computer-controlled linear actuator moves back and forth underneath the sample cuvette 110, causing the metal ball 275 to move in a commensurate manner. Clotting blood gradually impedes movement of the metal ball 275, eventually causing it to cease completely. A small-scale video camera 120 records time-dependent images indicating movement of the metal ball 275 that can be analyzed with an algorithm, as described above, to determine ACT. The small-scale video camera 220 is controlled by a circuit board 227 that includes a microprocessor, image-processing electronics, and other electronics for power management and other components. A first mounting component 237 supports both the circuit board 227 and small-scale video camera 220.

To make an optical spectroscopic measurement, similar to that shown in FIGS. 11 and 12, a circuit board 170 shaped like an annular ring supports three white-light LEDs 112a, 112b, 112c. The white-light LEDs 112a, 112b, 112c emit radiation ranging from about λ=380-1000 nm that fully illuminates the sample cuvette 110. The circuit board 270 is placed on top of the small-scale video camera 220 so that it surrounds a lens used therein. During a measurement, the circuit board 127 supplies power to the three white-light LEDs 112a, 112b, 112c, causing them to emit optical radiation in the spectral regions described above. The radiation passes through the optically transparent windows 117, 119 of the sample cuvette 110, where it is partially absorbed and scattered by the blood sample therein. The amount of optical absorption and scattering depends on the degree of clotting in the blood, and additionally any chemical reaction that may occur in the cuvette 110, e.g. due to an added reagent, such as an aptamer or aptamer/fluorophore compound. In these applications, a ‘baseline measurement’ is typically made before any reagent is added to the blood sample.

Radiation that passes through the sample cuvette and the blood within is then sensed with a photodetector 214 mounted on a separate circuit board (not shown in the figure), which is turn is supported by a second mounting component 231. Prior making an optical spectroscopic measurement of blood, the measurement component 100 first makes a baseline measurement wherein the above-described optical system measure a blood sample without any reagent (or alternatively an empty sample cuvette). This entails powering on the three white-light LEDs 112a, 112b, 112c with the circuit board 227 and detecting radiation with the photodetector. Once the baseline measurement is complete, the pipetting robot loads blood into the sample cuvette, where it first mixes with the clotting agent. The circuit board 227 then powers on the white-light LEDs 112a, 112b, 112c, which generate broadband optical radiation that passes through the sample cuvette and is detected by the photodetector 214. As described above, the front face of the photodetector 214 features computer-controlled optical filters that pass discrete, specific bands of radiation, each associated with optical wavelengths as indicated by the yellow squares in FIG. 11.

A series of setscrews 260, 261 allow the first 237 and second 231 mounting components to be laterally adjusted during manufacturing to improve collection of signals used for the imaging and optical spectroscopic measurements. In alternate embodiments, these setscrews 260, 261 are connected to computer-controlled actuators that move them in an automated manner. A secure mounting plate 101 holds all the above-mentioned components in place.

Referring to FIGS. 10A and 10B, the SSMD features a computer 69 and corresponding touchpanel display 77 that operates a graphical user interface 78. These systems, working in concert, allow a user (e.g. a cardiologist) to program in a panel of measurements, which the SSMD then automatically performs. They also allow display (e.g. time and frequency-domain plots) of data collected with the SSMD, and porting of these and other data to the hospital's EMR system.

### 5. Clinical Data

FIGS. 12A-12C show time-dependent waveforms measured using the multi-wavelength spectroscopy system from whole blood samples mixed with a clotting agent (Kaolin). The waveforms were measured from blood samples: 1) taken before the patient receives heparin (FIG. 12A); 2) taken after the patient receives heparin (FIG. 12B); and 3) taken after the patient receives both heparin and protamine, a medication used to reverse and neutralize the anticoagulant effects of heparin (FIG. 12C). All measurements were made with a programmable filter, disposed in front of the broadband photodetector, set to transmit radiation centered 2=630 nm with a bandwidth A of about 15 nm. The ACT corresponding to each blood sample was simultaneously measured with a gold-standard reference device (Hemochron), and is listed in the figures and additionally shown as a vertical dashed line. FIGS. 12D-12F show the first mathematical derivatives of the time-dependent waveforms in FIGS. 12A-12C; here, the horizontal dashed line corresponds to a ‘0’ value (i.e. when the time-dependent waveform stops changing), and the vertical dashed line again corresponds to the ACT value of the blood sample as measured with the gold-standard reference device.

As is clear from the time-dependent waveforms in FIGS. 12A-12C, transmission of radiation at λ=630 nm rapidly increases almost immediately after the whole blood samples are mixed with the clotting agent. Without being bound to any theory, this is likely to due to Kaolin enhancing the intrinsic clotting cascade. As blood clots, transmission of radiation near λ=630 nm gradually increases, likely due to a combination of decreasing optical absorption of compounds formed during the clotting cascade and an increase in light scattering of these compounds, which increase in size as blood clots. Both physiological mechanisms-decreased absorption and increased scattering-increases the amount of transmitted radiation that reaches the optical detector. As the clotting slows and reaches a maximum value, the rise in the time-dependent waveform begins to slow, and the transmitted radiation reaches a maximum value. For the pre-heparin (FIG. 12A) and protamine (FIG. 12C) samples, the transmission begins to gradually decrease once this maximum value is reached at, respectively, 191 and 184 seconds. Transmission of radiation from the heparinized sample (FIG. 12B) reaches a plateau for a short period of time near 283 seconds, and then gradually increases after that. This increase in ACT is due to heparin slowing the clotting process.

The derivatized signals shown in FIGS. 12D-12F indicate the rate of change of the time-dependent waveforms in FIGS. 12A-12C. The rate of change plateaus when these signals first cross a 0 value, as indicated in the figures by the dashed horizontal line. Stated another way, ACT calculated from the figures is indicated when the derivatized signal first crossed 0 and meets the dashed horizontal line. This time fiducial agrees well with the ACT value measured with the gold-standard, as indicated by the vertical line. Thus, software that detects a first 0-point crossing (i.e. when the derivatized signal evolves from a positive signal to a negative one) yields the ACT value. Such software, for example, would operate on a computational module within the measurement component.

FIGS. 13A-13F show time-dependent waveforms measured at the following optical wavelengths: 1) λ=415 nm (13A); 2) λ=480 nm (13B); 3) λ=515 nm (13C); 4) λ=555 nm (13D); 5) λ=630 nm (13E); and 6) λ=680 nm (13F). Waveforms for these figures were measured from whole blood samples, extracted from a patient treated with heparin and then protamine (e.g. similar to that for FIGS. 12C and 12F). As is clear from the figures, time dependent waveforms at λ=480, 515, and 555 nm continue to increase, even after the clotting time indicated by the dashed line (t=184 seconds). However, time dependent waveforms at λ=630 and 680 nm plateau at a time period consistent with the gold-standard ACT, with data measured at λ=630 showing the best agreement, thus indicating ideal measurement conditions.

While the optical transmission methodology described with reference to FIGS. 12 and 13 represents more of an indirect way to measure ACT, this parameter can also be measured directly using an imaging+mechanical system within the measurement component. FIGS. 14A and 14B show one embodiment of this system, where the digital camera (not shown) that is part of this system was used to take the images in figures. The sample cuvette 111 used to house blood for this measurement has square, parallel transparent windows, as opposed to the cylindrical cuvettes with curved windows shown in FIG. 2-4, but otherwise serves the same purpose. The cuvette 111 includes a whole blood sample, a collection of reflective, white beads 270, and a metal ball 275 whose motion is driven by an underlying linear actuator and magnet (not shown in the figure) as described above. The metal ball 275 serves two purposes: 1) when rapidly moved back and forth it can be used to mix the whole blood sample with a reagent, e.g. a clotting agent; and 2) it can be slowly driven to move in the blood sample to measure clotting. Surrounding the sample cuvette 111 are a first 129a and second 129b vibrator systems that, during a measurement, rapidly shake the cuvette and the white reflective beads 270, and the metal ball 275 therein. While this is happening, the digital camera periodically captures images of the cuvette, including the blood, reflective beads 270 and the metal ball 275 therein. Image-processing software operating on the computational module in the measurement component analyzes motion of these components during the clotting process; as shown in the figure, this software can use algorithms based on pattern recognition, AI, and/or ML to draw boxes around the metal ball 275 and one or more of the beads 277. By doing this, the software can track motions of these components during the clotting process. As blood clots, its viscosity gradually increases as it undergoes a phase transition from a liquid to a solid. This increase in viscosity impedes motion of the reflective beads 270, and a metal ball 275. Eventually, when blood within the cuvette 111 fully clots, both the reflective beads 270, and a metal ball 275 cease to move entirely. The computational model uses statistical methods, such as an ensemble average, to calculate the collective motion of these components. Such a model, for example, can reduce the collective motion of the components to a two-dimensional, time-dependent waveform (similar to those shown in FIG. 12) that can be analyzed to explicitly determine an absolute value of ACT.

Note that in FIG. 14A, which was measured before the first 129a and second 129b vibrator systems were activated, the reflective beads 270 were more or less evenly dispersed in the sample cuvette. During vibration, and in this particular example, the beads 270 tend to congregate in a corner of the cuvette 111. Although it is not clear from the two images shown in the figure, the collective motion of the beads 270 is also gradually impeded as the blood clots.

In embodiments, the indirect methodology for determining ACT, as described above with reference to FIGS. 12 and 13 can be combined with the relatively direct methodology for determining ACT, as described above with reference to FIGS. 14, to shorten the time it takes to complete a measurement. FIGS. 15A and 15B show an example of this. Referring first to FIG. 15A, the measurement component can make an initial measurement by simultaneously determining ACT from a whole blood sample within a sample cuvette by: 1) indirectly measuring a time-dependent optical wavelength at λ=630 nm with the optical system, as indicated is FIG. 12; and 2) directly measuring vibration-induced movement of reflective beads in the blood sample with the imaging+mechanical system, as indicated by FIG. 14. The direct measurement yields an absolute value of ACT that requires complete clotting of the blood; the time required for this is consistent with the full duration of the waveform shown in FIG. 15A, e.g. about 500 seconds. The indirect measurement yields information that can be analyzed with numerical techniques (e.g. curve fitting, AI, ML) to predict the absolute measurement, and in theory can be completed over a much shorter time period once the initial measurement is complete.

More specifically, and as shown in FIG. 15A, the initial measurement (“Measurement 1” in the figure) requires roughly 500 second to collect the complete time-dependent waveform (from the optical measurement) and the measure absolute ACT explicitly by analyzing the vibrating balls. Numerical techniques operating in software and run on the computational module then analyze the time-dependent waveform, e.g. by fitting it to a mathematical model. For subsequent measurements (“Measurements N, N+1” in the figure), as shown in FIG. 15B, the time-dependent waveform is again measured, but the computational model quickly analyzes just its initial portion (e.g. the first 100 seconds, as indicated by the gray box in the figure), and uses this information to predict the eventual ACT. For example, the two waveforms shown in FIG. 15B have relatively slow rise times compared to that in FIG. 15A, indicating that they are associated with longer ACT values (221 and 258 seconds, respectively). Using this approach, ACT can be predicted relatively early on, e.g. after about 100 seconds; as the system collects more data points for an individual waveform, this prediction can be updated (presumably with increased accuracy) until clotting is complete and the time-dependent waveform reaches a plateau, as shown in FIG. 12.

In embodiments, the measurement described with respect to FIGS. 15A and 15B can be done with any of the approaches described herein, e.g. using time-dependent impedance/reactance waveforms, ensemble averages measured with the imaging+mechanical system, and other waveforms.

As described above, the SSMD provides three unique approaches for measuring ACT: 1) time-resolved optical absorption; 2) time-resolved imaging of a mechanical event (a moving metal ball controlled by stationary or moving magnets); and 3) time-resolved electrical impedance or reactance spectroscopy, using either a single frequency or multiple frequencies of injected electrical current. Additionally, simply capturing an image of clotting blood within the sample cell with the video camera described above, and then analyzing these results with image processing and an associated algorithm, may yield ACT. This is because the physical structure of blood changes as it clots over time, and this is likely manifested as a change in the associated image of the sample cell.

The multiple measurements of ACT made by the SSMD may be collectively processed in a variety of ways to yield a single, cumulative measurement. For example, they can be averaged together, with any ACT value falling outside a pre-determined region (e.g. more than 1 standard deviation from the mean ACT value) being discarded. Or the average or, alternatively, a weighted average can be calculated, with ACT values determined from time-dependent signals that fail to meet pre-determined thresholds for noise discarded from the average. This could occur, for example, if blood within the sample cuvette coagulated in a particularly non-uniform manner, thereby indicating the measurement technique used to determine ACT in this instance would be flawed. ACT is known to be somewhat device-specific (e.g. the prior art devices may yield different values according to the algorithms used therein). Thus, future clinical studies may indicate that one approach used by the SSMD (e.g. time-resolved optical absorption) may correlate better with a particular device (e.g. the i-STAT 1, manufactured by Abbot), while another (e.g. time-resolved electrical impedance) may correlate better with another device (e.g. the ACT Plus, manufactured by Medtronic). Here, the SSMD could report multiple ACT values, e.g. an ‘Abbot value’ that is more consistent with Abbot's measurement, and a ‘Medtronic value’ that is more consistent with Medtronic's value. In general, the SSMD is designed to report multiple ACT values.

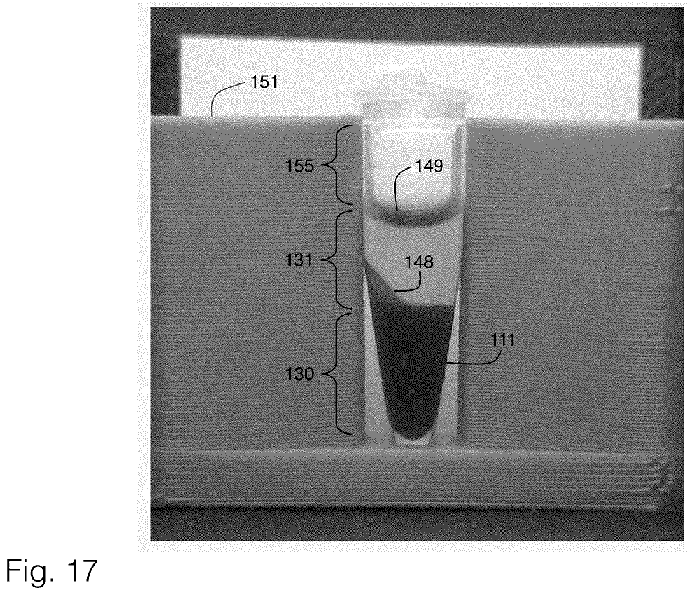

As described above, the digital camera within the measurement component captures images of post-centrifuged samples, and analyzes these images to accurately measure hematocrit. FIG. 17 shows such an image of a sample cuvette 111, filled with whole blood and secured by a sample holder 151 within the measurement component. The image was taken by the digital camera and clearly shows whole blood within the sample cuvette 111 separated into red blood cells (portion 130) and plasma (portion 131) after being centrifuged. To determine values of hematocrit, image-processing software operating on the measurement component determines volumes associated with portions 130, 131, and then uses these to calculate hematocrit (the ratio of the volume of red blood cells to the total volume of blood) as described above. To increase accuracy of the measurement, the image-processing software may be augmented with more sophisticated computational techniques, e.g. machine learning, artificial intelligence, and/or optical pattern recognition.

To ensure the highest degree of accuracy for hematocrit measurements, the image-processing software must account for artifacts in the images that occur after centrifugation. For example, in FIG. 17 a top portion 155 of the sample cuvette 111 is filled with air, resulting in a crescent-shaped ‘meniscus’ 149 at the plasma/air interface that can complicate processing of the image. During image processing, the meniscus 149 can be numerically analyzed with an algorithm, (e.g. one based on numerical fitting or machine learning) to account for its shape and accurately estimate the corresponding volume of plasma. Alternatively, because plasma consists mostly of water, mixing the whole blood with a liquid with a density lower than water (e.g. isopropyl alcohol), and then centrifuging the sample, removes the meniscus at the plasma/air interface, thereby simplifying analysis of the image and improving accuracy of the hematocrit measurement. Additionally, as shown in the image, centrifugation causes a thin film of blood 148 to form near the red blood cell/plasma interface. When the image is processed with the image-processing software, this artifact can be accounted for using simple assumptions (e.g. the red blood cell/plasma interface is necessarily a straight line) or alternatively by using more sophisticated processing tools, e.g. machine learning.

To evaluate this measurement approach, a clinical study was performed wherein blood samples were extracted from patients. Whole blood from each patient was separated into first and second components, wherein the second component was centrifuged to separate blood cells from plasma. The plasma from the second component was then used to titrate whole blood in the first component to systematically vary the hematocrit. Each titrated whole blood sample was then placed in a unique sample cuvette and measured with the measurement component as described above, i.e. centrifuged, imaged with the digital camera, and then analyzed with image-processing software. Prior to being centrifuged, blood from each sample cuvette was also measured with a POC device that yielded both hematocrit and hemoglobin. Finally, both hematocrit and hemoglobin were measured with gold-standard reference techniques that accounted for the volume of titrated plasma along with untitrated volumes of both red blood cells and plasma.

FIGS. 18A and 18B are scatter plots showing hematocrit values measured with the reference technique and compared to, respectively, hematocrit values measured by the measurement component by processing images similar to those shown in FIG. 17 and hematocrit values measured with the POC device. The dashed line in the figure (y=x) indicates perfect correlation between the two values. Given the quality of images like that shown in FIG. 17—which show both plasma and red blood cells that are clearly and entirely separated—it is assumed that hematocrit measured with the measurement component has an extremely high degree of accuracy, e.g. likely higher than the reference technique. Thus, any deviation from perfect correlation in the plot in FIG. 18A is likely due to errors in the reference device. As is clear by comparing FIGS. 18A and 18B, for measurements of hematocrit, when compared to the reference technique, performance of the measurement component (r²=0.79) as shown in FIG. 18A is superior to performance of the POC device (r²=0.51) as shown in FIG. 18B.

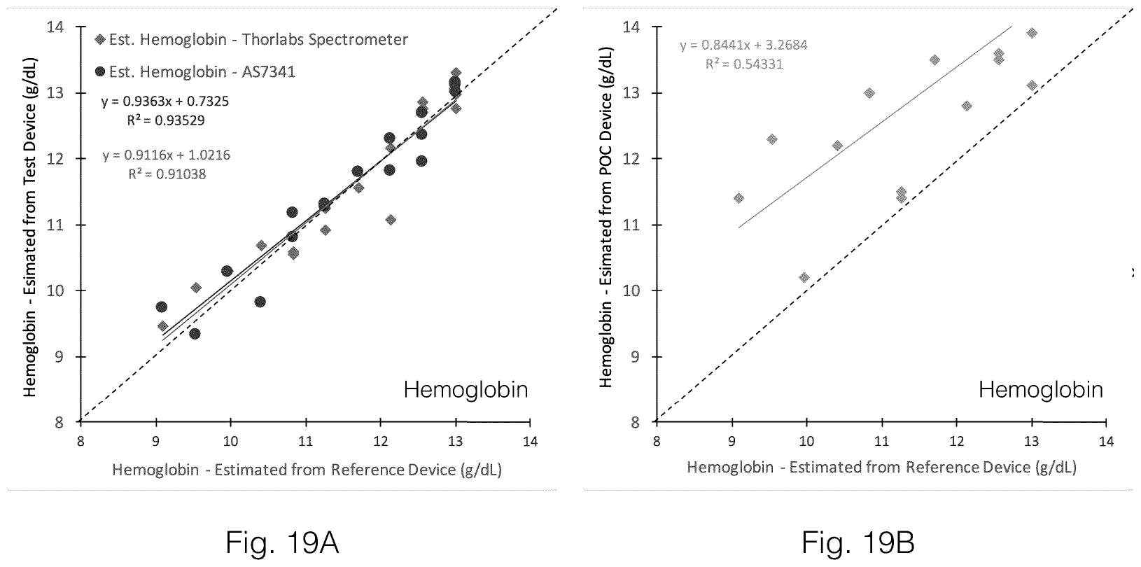

A similar clinical trial was used to characterize the measurement component's measurement of hemoglobin, which is made using optical spectroscopy, as described above. For this trial, the measurement component was outfitted with two different optical spectrometers: 1) a high-end and relatively expensive system from Thorlabs; and 2) a low-cost system featuring the AMS AS7341. These systems, along with the POC device described above, measured hemoglobin from the titrated blood samples. Results from these measurements were then compared to those from the reference technique. FIGS. 19A and 19B are scatter plots showing the results of this analysis. As before, the dashed line in the figure (y=x) indicates perfect correlation between the two values. The figures clearly show that when compared to the reference technique for measurements of hemoglobin, performance of the measurement component using either the Thorlabs spectrometer (r²=0.91) or the AS7341 (r²=0.94) as shown in FIG. 19A are both superior to performance of the POC device (r²=0.54) as shown in FIG. 19B. Additionally, these results indicate that for measuring hemoglobin, the low-cost AS7341 shows performance that is the same and possibly better than the high-end Thorlabs spectrometer. This indicates that the AS7341 can be effectively used in the measurement component for measurements of hemoglobin, and also likely for other measurement described above that rely on optical spectroscopy.

In other embodiments, PTT or aPTT may be calculated alongside of ACT, and these two parameters are then processed algorithmically together. As described above, PTT (or aPTT) is widely considered to be a more accurate metric for coagulation, but is rarely used in the Cath Lab because of the time-consuming and inconvenient nature of the test used to determine it. In embodiments, PTT/aPTT may be determined initially for a patient; ACT is then determined independently using each of the three techniques described herein (optical, impedance, mechanical/imaging). The ACT test that best agrees with the PTT/aPTT measurements is then used going forward.

In all cases, the microprocessor controlling the SSMD may run computer code that utilizes different signal-processing techniques to analyze signals measured by the SSMD's various sensors to extract ACT values. In embodiments, for example, the computer code may run numerical ‘fitting’ algorithms to analyze time-dependent waveforms, such as the optically generated waveform in FIG. 12, or the impedance-generated waveform in FIG. 21D, to extract the ACT values. For the fitting algorithm, a pre-determined mathematical function is derived from a physiological process that describes the blood clotting, e.g. a linear function coupled with an exponential function. The mathematical function is then input into computer code that runs the fitting algorithm, which then iteratively varies parameters associated with the function (e.g. an exponential time constant associated with a non-linear function; a slope and y-intercept associated with a linear function) until the pre-determined mathematical function best matches the time-dependent waveform measured by the SSMD. The computer code then calculates ACT values from the ‘fit’ and its associated parameters.

### 6. Other Embodiments

Other embodiments are within the scope of the invention. For example, the SSMD can be coupled to a physiological monitoring device, such as a wearable device (e.g. a patch, ring, wristband featuring embedded physiological sensors), to better characterize a patient during a procedure or post-surgery recovery. In this embodiment, clinical data characterizing the patient's vital signs and hemodynamic parameters, as measured with the monitoring device, can be combined with clinical data characterizing the patient's blood (e.g. ACT, hemoglobin) to predict patient decompensation.

In embodiments, for example, clinical data from both sources can be used to predict sepsis during post-surgery recovery. Markers for sepsis-related decompensation include increased lactic acid; this can be monitored using the SSMD and a reagent containing an aptamer that specifically binds to this biomarker. Examples of such aptamers that bind to lactic acid include those described in the following references, the contents of which are incorporated herein by reference: 1) Frith et al., ‘Towards development of aptamers that specifically bind to lactate dehydrogenase of Plasmodium falciparum through epitopic targeting’, Malar J. 2018; 17:191 (published online May 3, 2018 doi: 10.1186/s12936-018-2336-z); and 2) Minagawa et al., ‘Modified DNA Aptamers for C-Reactive Protein and Lactate Dehydrogenase-5 with Sub-Nanomolar Affinities’, (published online Apr. 13, 2020, doi: 10.3390/ijms21082683). When coupled to a molecule that can be detected with one of the approaches described above, e.g. an assay using the small-scale camera to detect a colorimetric change, these aptameric compounds, or comparable derivatives thereof, can detect the concentration of lactic acid in the patient's blood. This value can then be collectively processed with physiological signals—such as an increase in heartrate or blood pressure, or a decrease in stroke volume—to determine a level of decompensation in the septic patient.

In a related embodiment, aptamers used to detect BNP can be combined with physiological signals in a manner similar to that described above to determine a patient's progressing towards congestive heart failure (herein “CHF”). Examples of such aptamers that bind to BNP include those described in the following references, the contents of which are incorporated herein by reference: 1) Grabowska et al. ‘Electrochemical Aptamer-Based Biosensors for the Detection of Cardiac Biomarkers’, ACS Omega 2018, 3, 12010-12018 (published online Sep. 26, 2018, doi: 10.1021/acsomega.8b01558); and 2) Bruno et al., ‘Preliminary Development of a DNA Aptamer-Magnetic Bead Capture Electrochemiluminescence Sandwich Assay for Brain Natriuretic Peptide’, Microchem J. 2014 Jul. 1; 115:32-38 (published online Jul. 1, 2015, doi: 10.1016/j.microc.2014.02.003). The contents of each of these documents are incorporated herein by reference.

An increase in BNP indicates a patient's progression towards CHF. Likewise, this condition is typically associated with a decrease in the patient's stroke volume, and increase in heart rate, and an increase in respiration rate. As with sepsis, levels of BNP in the patient's blood, as determined with the SSMD, can be combined with changes in these physiological signals to determine the patient's progression towards CHF.

In embodiments, analytical models based on AI and/or ML can collectively process SSMD-determined proteins in the patient's blood with their physiological signals to determine the patient's disease state.

In embodiments, blood and reagents can be mixed (e.g. prior to being loaded in the sample cuvette, or in the actual sample cuvette) with a mechanical mixing component. This component can be, for example, a propeller-shaped device or something similar coupled to a rotary motor. Such a system would replace the magnetically controlled metal ball as used for mixing purposes, as described above. Here, because the metal ball is not present, both the optical and impedance-based approaches described above are used to measure ACT or PTT; the mechanical measurement featuring the camera is typically not used. Alternatively, the camera can collect time-dependent images of the propeller-shaped device while it is rotating, analyze these to determine a two-dimensional time-dependent signal, and then process this signal with the algorithms described above to determine ACT or PTT.

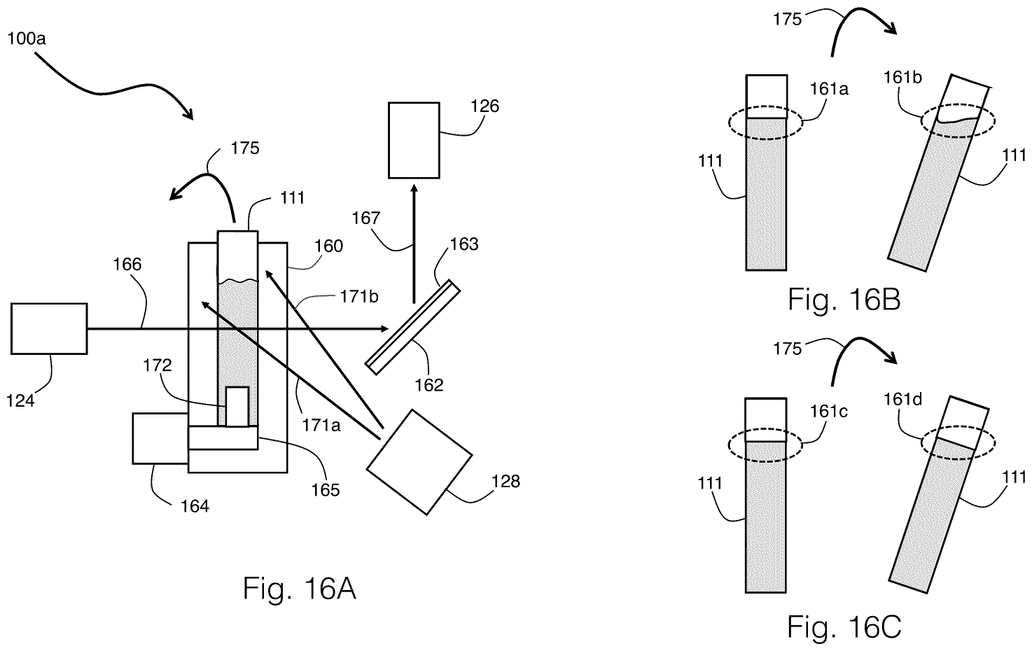

In other embodiments, the measurement and blood-extraction components within the SSMD can feature other configurations. For example, referring to FIGS. 16A-16C, a measurement system 100a according to an alternate embodiment of the invention features optical imaging+mechanical (component 66 in FIG. 4) and multi-wavelength spectroscopy (component 63 in FIG. 4) systems that are combined and modified compared to those described above. In FIG. 16A, for example, the multi-wavelength spectroscopy system features an LED 124 that emits white light as indicated by arrow 166 that passes through a sample holder 160 and a blood-filled cuvette 111. The white light is partially absorbed by the blood within the cuvette 111, and the reflects off a reflecting film 163 disposed on a surface of a glass mirror 162. The reflecting film 63 can be, for example, a partial reflector. The mirror 162 directs the reflected radiation, as indicated by arrow 167, into a multi-wavelength photodetector 126, where it is then measured as described herein to generate time and frequency-dependent waveforms indicating clotting. Simultaneously, a digital camera 128 images the blood-filled cuvette 111 as indicated by arrows 171a, 171b, and particularly an area of the cuvette 111 where a ‘blood/air interface’ is present. The blood/air interface is where an upper portion of blood in the cuvette is exposed to air outside of the cuvette. A motor 164 connects to a moving arm 165, which in turn attaches to a band 172 that connects to the cuvette 111.

During a measurement process, the motor 164 rotates and rocks the moving arm 165, which in turn causes the cuvette 111 to slowly swing back and forth, as indicated by arrow 175. This motion causes blood within the cuvette to slosh back and forth in a consistent manner, depending on its degree of clotting.

Referring to FIG. 16B, when the cuvette 111 is filled with unclotted blood and is in a vertical position, the blood/air interface as indicated by dashed circulate 161a is relatively flat. When the motor 164 and moving arm 165 rotate the cuvette 111 to an angled position, the unclotted blood in the blood/air interface as indicated by dashed circle 161b shifts to one side due to the liquid nature of the blood.

Referring to FIG. 16C, when the cuvette 111 is filled with clotted blood and in a vertical position, the blood/air interface as indicated by dashed circle 161c is again relatively flat, similar to that shown in FIG. 16B. However, when the motor 164 and moving arm 165 rotate the cuvette 111 to an angled position, the clotted blood in the blood/air interface as indicated by dashed circle 161d shifts does not move or slosh in any way, and the interface remains a flat line. Periodically repeating this process while simultaneously using the digital camera to image the blood/air interface, when coupled with software that performs pattern recognition, allows the optical imaging+mechanical system to image the sloshing of the blood in the blood/air interface, thereby yielding a direct measurement of clotting time. Such a clotting time occurs when the blood/air interface is a solid, slosh-free line, as shown in by the dashed circle 161d.

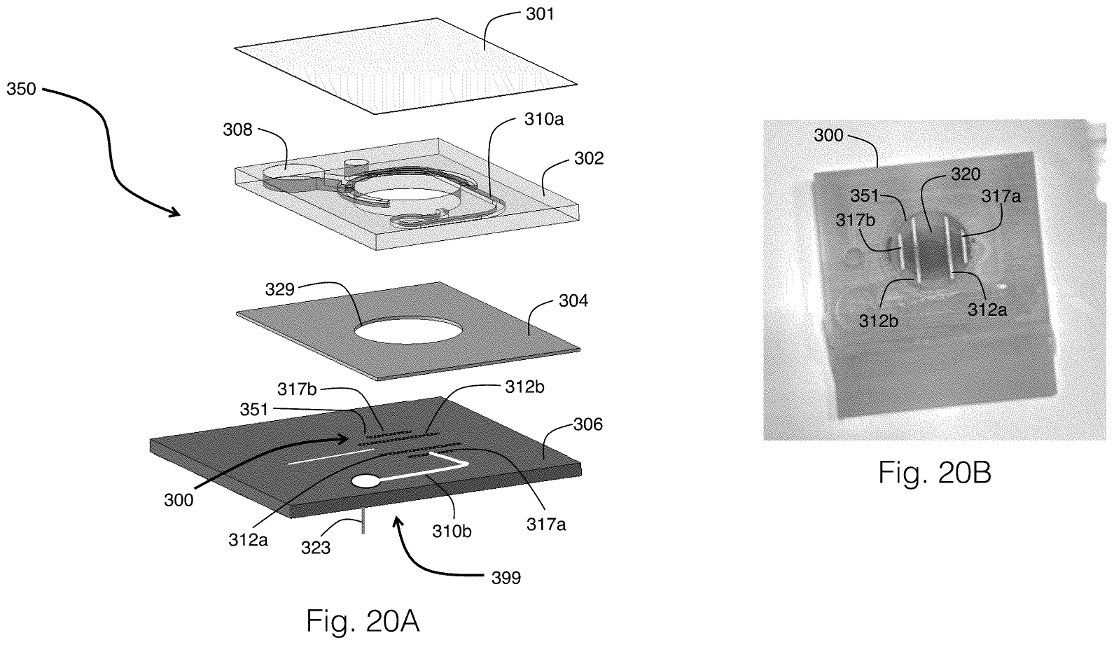

In related embodiments, for example, as shown in FIGS. 20A-20B, the SSMD can take the form of a small-scale, wearable patch 350. Like the SSMD described herein, the wearable SSMD patch 350 features a blood-extraction component 399 and a measurement component 300. The blood-extraction component 399 connects to a small-scale catheter 323 (e.g. a needle or filament) that, during use, presses into the patient's skin an extracts a small volume of blood or interstitial fluid. The catheter 323 connects to a microfluidics channel 310b that is formed from an underlying printed circuit board (herein “PCB”) substrate 306 attached to an overlying plastic cover 302 by an adhesive layer 304. The adhesive layer 304 includes a circular opening 329 so that, when the PCB substrate 306 and plastic cover 302 attach, the resultant structure forms a small ‘well’ 351, fed by the microfluidics channel 310a, 310b, for blood to pool, clot over time, and be subsequently measured. For this to happen, the plastic cover includes an opening 308 that facilitates the draw of blood through the microfluidics channel 310a, 310b and into the well 351 via capillary action. A top cover 301 forms a barrier above the plastic cover 302 to prevent oxygen from exposing the blood within the well 351.

The well 351 includes sense 312a, 312b and drive 317a, 317b electrodes that, once connected to an internal impedance circuit, make a measurement as described above. During this measurement, the drive electrodes 317a, 317b, which are located distally, inject high-frequency, low-amperage current into the well 351, which in turn samples the clotting blood as described above. The sense electrodes 310a, 310b, which are located internally, measure the electrical impedance encountered by the injected current in the clotting blood. FIGS. 21A-21D indicate this process in more detail. As shown by the image in FIG. 21A and plot in FIG. 21D, for this measurement, the impedance starts out at a relatively high level as blood enters the well. At t=450 s, as shown by the image in FIG. 21B, the blood begins to clot and electrical impedance measured by the measurement component 300 begins to drop rapidly, indicating that the blood's conductivity in the well 351 increases during coagulation. Eventually at around t=750s the blood fully clots, as indicated by the image in FIG. 21C, and the impedance stays relatively constant. The time-dependent graph in FIG. 21D indicates the temporal evolution of this coagulation process. During a measurement, a microprocessor within SSMD patch 350 processes signals like this to determine ACT.

In embodiments, components used in the SSMD patch 350 shown in FIGS. 20A-20B can be incorporated into form factors that are more easily worn on the patient's body, e.g. a form factor resembling a watch, ring, or bracelet.

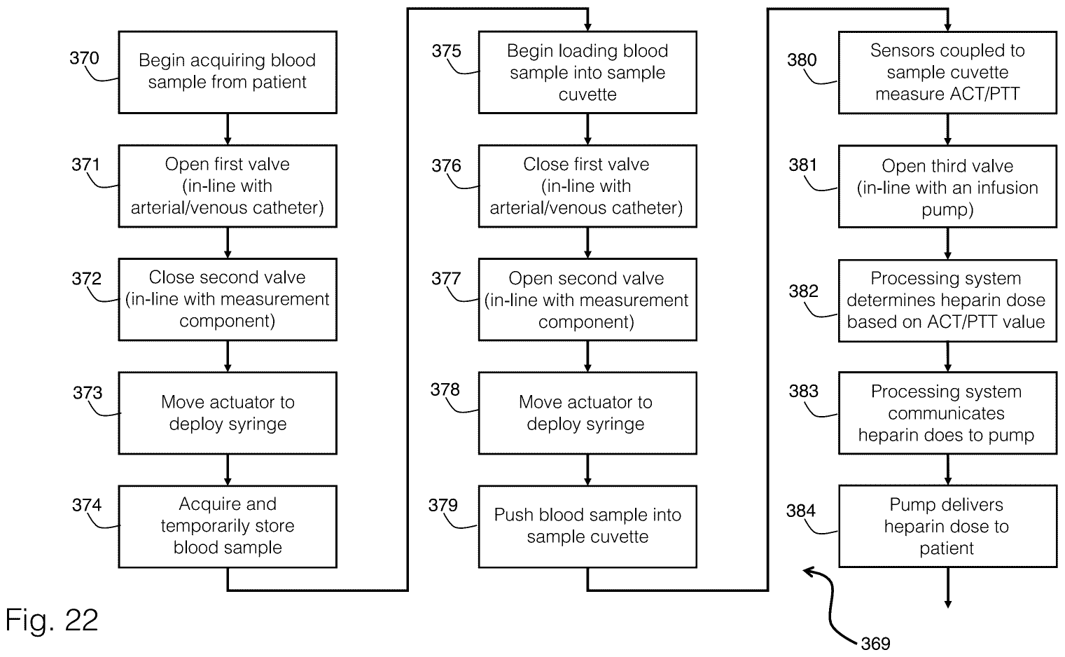

In other embodiments, as indicated by the flow chart in FIG. 22, the SSMD can be used in concert with an infusion pump to deliver heparin to a patient in a closed-loop process 369. Here, the SSMD's blood-removal component can feature two valves, and integrates with a heparin-delivery system, e.g. one that includes heparin (e.g. dissolved in a saline bag) an infusion pump, and a third value.

The closed-loop process 369 starts as the SSMD begins to acquire blood from the patient (step 370). To do this, its processing system opens a first valve in-line with a catheter disposed in the patient's arterial or, more preferably, venous system (step 371), and closes a second valve in-line with the measurement component and the sample cuvette therein (step 372). The processing system then controls an actuator to deploy a syringe (step 373), which in turn extracts blood from the patient and temporarily stores it (step 374), e.g. in tubing in contact with the catheter.

Once the blood is stored (step 374), the processing system begins to load it into the sample cuvette (step 375) by closing the first valve in-line with the patient's arterial/venous system (step 376) and opening the second valve in-line with the measurement component (step 377). The processing system then controls the same actuator used in step 373 to push the blood sample into the sample cuvette (step 379) within the SSMD's measurement component.

The measurement component, using one (or all) of the above-described optical, impedance, and mechanical sensors coupled to the sample cuvette, measures blood within the sample cuvette to determine a corresponding ACT/PTT value (step 380). Based on this value, the processing system opens a third valve, separate from the blood-extraction component, that is in-line with an external infusion pump (step 381). The infusion pump may couple to the patient using the same catheter used in the blood-extraction component, or with a separate catheter. An algorithm running on the processing system uses the ACT/PTT value to first determine if the patient needs heparin, and if so how much (step 382). Here, the algorithm can be a simple look-up table incorporating pre-determined values of ACT/PTT values and corresponding heparin doses; the pre-determined values, for example, can be determined using clinical studies, EMR records, and values reported in the medical literature. In related embodiments, the algorithm additionally considers biometric data from the patient (e.g. their weight, age, gender, medical condition, typically determined from the EMR) to determine the heparin dose. Once it is determined, the processing system communicates the heparin dose to the infusion pump (step 383), e.g. through a wired or wireless interface, which then delivers it to the patient (step 384).

The closed-loop process 369 can be deployed with systems not worn on the patient (e.g. similar to those shown in FIG. 5), or with a completely wearable system (e.g. similar to that shown in FIG. 17).

Still other embodiments are within scope of the invention. For example, the system described above can also include additional sensors for measuring other properties for human blood. The system can also include a three-axis digital accelerometer and a temperature sensor to measure from the patient, respectively, three time-dependent motion waveforms (along x, y, and z-axes) and temperature values within the SSMD. The optical sensor can include a heating element featuring a thin Kapton® film with embedded electrical conductors arranged in a serpentine pattern to increase perfusion and, subsequently, the strength optical signals.

In other embodiments, the system described above can be used for other surgical applications, or to remotely from blood-based parameters from a patient at home. In this latter case, the system may be coupled to a cloud-based system that may also include AI and ML algorithms to process information.

In other embodiments, the entire SSMD may be heated (e.g. to) 98.6° to better match the temperature of the human body.

The reagent used for the ACT measurement is typically celite or Kaolin, which activates a clotting reaction. Kaolin activates the intrinsic clotting pathway, thereby leading to the activation of a thrombin substrate that is not fibrinogen. In other embodiments, the SSMD includes an electrochemical sensor that indicates when the activation of a thrombin substrate is complete. The substrate used in this assay (H-D-phenylanyl-pipecolyl-arginine-p-amino-p-methoxydiphenylamine) mimics the thrombin-cleaved amide linkage found in fibrinogen. The product of the reaction consists of an electrochemically inert tripeptide (phenylalanyl-pipecolyl-arginine) and an electroactive compound (NH₃⁺—C₆H₄—NH—C₆H₄—OCH₃). The electroactive compound, NH₃⁺—C₆H₄—NH—C₆H₄—OCH₃, is detected amperometrically.

In other embodiments, measurements made by the SSMD may be free of any clotting reagent. For example, the impedance measurement used by the SSMD can yield the blood's viscosity, which is related to ACT by a pre-determined linear regression formula. This approach has an important advantage that it can be done very rapidly (e.g. in a matter of seconds), making quasi-continuous measurements of ACT a reality. In still other embodiments, a ‘hybrid’ measurement is made where the reagent-based ACT measurement is performed with a first electrode to yield an initial ‘ACT calibration’. The system then makes the above-described measurements of viscosity with a reagent-free electrode, and combines these with the ACT calibration to yield accurate (and rapid) follow-on measurements of ACT.

Clinical studies supporting the idea of using electrical impedance to measure the viscosity of a fluid (most notably human blood) are described in the following references, the contents of which are incorporated herein by reference: Pop et al., ‘On-line electrical impedance measurement for monitoring blood viscosity during on-pump heart surgery’, Eur Surg Res. 2004; 36 (5): 259-265 and Berney et al., ‘Impedance measurement monitors blood coagulation’, ADI. 2008; 42 (3): 42-08. Relative changes in blood viscosity, in turn, indicate corresponding changes in Heparin levels and ACT, as described in the following references, the contents of which are incorporated herein by reference: Hitosugi et al., ‘Changes in blood viscosity by heparin and argatroban’, Thromb Res. 2001; 104 (5): 371-374. and Ranucci et al., ‘Blood viscosity during coagulation at different shear rates’, Physiol Rep. 2014; 2 (7): e12065. Published 2014 Jul. 3. doi: 10.14814/phy2.12065. This indicates that electrical impedance represents a potential measurement for determining relative changes in ACT in blood without using reagents.

Likewise, visible and near-infrared transmission optical spectroscopy is well established for determining hemoglobin concentration from blood samples processed with clotting reagents as describe in the following references, the contents of which are incorporated herein by reference: Whitehead et al., ‘Methods and analyzers for hemoglobin measurement in clinical laboratories and field settings’, Ann N Y Acad Sci. 2019; 1450(1):147-171. Recent work indicates that relative changes in these parameters can yield results using comparable optical techniques and no reagents, as described in the following references, the contents of which are incorporated herein by reference: Zhang et al., ‘Nondestructive Measurement of Hemoglobin in Blood Bags Based on Multi-Pathlength VIS-NIR Spectroscopy’, Sci Rep. 2018; 8 (1): 2204. Published 2018 Feb. 2. doi:10.1038/s41598-018-20547-2.

The system above is described for use with human patients. In other embodiments, the system can be used with animals for veterinary applications. Such a system, for example, is typically a wearable system, as described above.

These and other embodiments of the invention are deemed to be within the scope of the following claims.

What is claimed is:

1. A system for predicting activated clotting time (ACT) from a blood sample within a sample holder, comprising:
a first ACT-measuring system configured to measure a mechanical property indicating clotting of the blood sample;
a second ACT-measuring system configured to measure a first time-dependent waveform and a second time-dependent waveform that are both affected by clotting of the blood sample; and,
a processing system configured to perform the following steps: 1) analyze the mechanical property to determine a first value of ACT; 2) analyze both the first time-dependent waveform and the first value of ACT to determine a model for predicting ACT; and 3) using the model, analyze the second time-dependent waveform to predict a second value of ACT.

2. The system of claim 1, wherein the first ACT-measuring system comprises a digital camera configured to image the blood sample and a mechanical system configured to move the sample holder.

3. The system of claim 2, wherein the mechanical system comprises a vibrator system coupled to the sample holder.

4. The system of claim 3, wherein step 1) performed by the processing system comprises analyzing motion of blood clots within the sample holder to determine the first value of ACT.

5. The system of claim 4, wherein step 1) performed by the processing system comprises using an algorithm comprising at least one of pattern recognition, machine learning, and artificial intelligence to analyze motion of blood clots within the sample holder.

6. The system of claim 3, wherein the sample holder comprises reflective beads mixed with the blood sample.

7. The system of claim 5, wherein step 1) performed by the processing system comprises analyzing motion of the reflective beads to determine the first value of ACT.

8. The system of claim 7, wherein step 1) performed by the processing system comprises using an algorithm comprising at least one of pattern recognition, machine learning, and artificial intelligence to analyze motion of the reflective beads.

9. The system of claim 2, wherein the mechanical system comprises a motorized system connected to the sample holder and configured to move the sample holder.

10. The system of claim 9, wherein the motorized system is configured to rock the sample holder back and forth.

11. The system of claim 10, wherein the digital camera is configured to collect images of blood moving within the sample holder.

12. The system of claim 11, wherein step 1) performed by the processing system comprises analyzing motion of the blood within the sample holder to determine the first value of ACT.

13. The system of claim 10, wherein the digital camera is configured to collect images of a blood/air interface within the sample holder.

14. The system of claim 13, wherein step 1) performed by the processing system comprises analyzing the blood/air interface to determine the first value of ACT.

15. The system of claim 14, wherein step 1) performed by the processing system comprises using an algorithm comprising at least one of pattern recognition, machine learning, and artificial intelligence to analyze the blood/air interface.

16. The system of claim 1, wherein the second ACT-measuring system comprises an optical system.

17. The system of claim 16, wherein the optical system comprises a light source and a photodetector.

18. The system of claim 17, wherein the light source is positioned on one side of the sample holder, and the photodetector is positioned on an opposing side of the sample holder.

19. The system of claim 18, wherein the light source and photodetector are further configured to measure time-dependent optical absorption of the blood sample to determine the first time-dependent waveform.

20. The system of claim 19, wherein step 2) performed by the processing system comprises analyzing the time-dependent optical absorption of the blood sample and the first value of ACT to determine the model for predicting ACT.

21. The system of claim 20, wherein step 2) performed by the processing system comprises using an algorithm comprising at least one of numerical fitting, pattern recognition, machine learning, and artificial intelligence to analyze the blood/air interface.

22. The system of claim 17, wherein the light source and the photodetector are positioned on the same side of the sample holder.

23. The system of claim 22, wherein the light source and photodetector are further configured to measure time-dependent optical reflectance of the blood sample to determine the first time-dependent waveform.

24. The system of claim 23, wherein step 2) performed by the processing system comprises analyzing the time-dependent optical absorption of the blood sample and the first value of ACT to determine the model for predicting ACT.

25. The system of claim 24, wherein step 2) performed by the processing system comprises using an algorithm comprising at least one of numerical fitting, pattern recognition, machine learning, and artificial intelligence to analyze the blood/air interface.

26. The system of claim 1, wherein the second ACT-measuring system comprises an impedance/reactance system.

27. The system of claim 26, wherein the impedance/reactance system comprises a sense electrode and a drive electrode.

28. The system of claim 27, wherein the drive electrode is configured to inject electrical current into the blood sample, and the sense electrode is configured to measure a voltage that is a function of the injected electrical current.

29. The system of claim 28, wherein the impedance/reactance system is further configured to measure time-dependent electrical impedance of the blood sample to determine the first time-dependent waveform.

30. The system of claim 29, wherein step 2) performed by the processing system comprises analyzing the time-dependent electrical impedance of the blood sample and the first value of ACT to determine the model for predicting ACT.

31. The system of claim 30, wherein step 2) performed by the processing system comprises using an algorithm comprising at least one of numerical fitting, pattern recognition, machine learning, and artificial intelligence to analyze the blood/air interface.

32. The system of claim 29, wherein the impedance/reactance system is further configured to measure time-dependent electrical reactance of the blood sample to determine the first time-dependent waveform.

33. The system of claim 32, wherein step 2) performed by the processing system comprises analyzing the time-dependent electrical reactance of the blood sample and the first value of ACT to determine the model for predicting ACT.

34. The system of claim 33, wherein step 2) performed by the processing system comprises using an algorithm comprising at least one of numerical fitting, pattern recognition, machine learning, and artificial intelligence to analyze the blood/air interface.

35. A system for predicting activated clotting time (ACT) from a blood sample within a sample holder, comprising:
a first ACT-measuring system comprising an imaging system configured to measure a mechanical property indicating clotting of the blood sample;
a second ACT-measuring system comprising an optical system configured to measure a first time-dependent waveform and a second time-dependent waveform that are both affected by clotting of the blood sample; and,
a processing system configured to perform the following steps: 1) analyze the mechanical property to determine a first value of ACT; 2) analyze both the first time-dependent waveform and the first value of ACT to determine a model for predicting ACT; and 3) using the model, analyze the second time-dependent waveform to predict a second value of ACT.

36. A system for predicting activated clotting time (ACT) from a blood sample within a sample holder, comprising:
a first ACT-measuring system comprising an imaging system configured to measure a mechanical property indicating clotting of the blood sample;
a second ACT-measuring system comprising an impedance/reactance system configured to measure a first time-dependent waveform and a second time-dependent waveform that are both affected by clotting of the blood sample; and,
a processing system configured to perform the following steps: 1) analyze the mechanical property to determine a first value of ACT; 2) analyze both the first time-dependent waveform and the first value of ACT to determine a model for predicting ACT; and 3) using the model, analyze the second time-dependent waveform to predict a second value of ACT.
